(19) United States
(12) Patent Application Publication
(10) Pub. No.: US 2025/0197843 A1
(43) Pub. Date: Jun. 19, 2025

(54) INSTRUMENT AND METHODS INVOLVING HIGH-THROUGHPUT SCREENING AND DIRECTED EVOLUTION OF MOLECULAR FUNCTIONS

(71) Applicant: University of Florida Research Foundation, Incorporated, Gainesville, FL (US)

(72) Inventors: Jing PAN, Gainesville, FL (US); Yongchen TAI, Gainesville, FL (US)

(21) Appl. No.: 18/847,847

(22) PCT Filed: Apr. 4, 2023
(86) PCT No.: PCT/US2023/017447

§ 371 Date: Sep. 17, 2024

Related U.S. Application Data
(60) Provisional application No. 63/362,702, filed on Apr. 8, 2022.

(51) Int. Cl. C12N 15/10

(52) U.S. Cl. CPC C12N 15/1062; C12N 15/1065

(57) ABSTRACT

Apparatuses and corresponding methods are provided for determining molecular properties of a molecule. To determine molecular properties of the molecule, a molecule library comprising the molecule is generated, a focused library comprising the molecule is generated by filtering the molecule library; the focused library is attached to a flow cell of the imaging instrument; and a fluid control module of the imaging instrument is controlled to incubate the flow cell with a fluid comprising labelled target molecules. A light source module and an imaging module of the imaging instrument are operated in a selected operational mode such that the light source module illuminates the flow cell and the imaging module captures light generated by fluorescence by the labelled target molecules within the flow cell. Image data is generated based on the captured light and the imaging data is analyzed to determine molecular properties of the at least one molecule.

### CROSS-REFERENCE TO RELATED APPLICATIONS

This application claims priority to U.S. Application No. 63/362,702, filed Apr. 8, 2022, the content of which is hereby incorporated by reference in its entirety.

### STATEMENT OF GOVERNMENT INTEREST

The invention was made with government support under grant number R21 EB032455 awarded by the National Institutes of Health. The government has certain rights in the invention.

### TECHNICAL FIELD

Various embodiments relate to sequencing and screening of molecules while maintaining genotype-phenotype linkage. For example, various embodiments relate to kinetic and/or non-equilibrium screening of molecules while maintaining genotype-phenotype linkage for the molecules. For example, various embodiments relate to an instrument for imaging molecules and/or interactions between molecules and a sample while maintaining genotype-phenotype linkage for the molecules.

### BACKGROUND

Molecule display is a group of techniques where molecules, such as proteins and peptides, are presented or displayed on the surface of a cell, a virus (cell surface display or phage display), or a biomolecule, such as a ribosome (ribosome display) or mRNA (mRNA display). The displayed library of molecules can be screened for desired functions or properties. mRNA display exploits a covalent chemical bond to fuse protein and its encoding mRNA during translation. Ribosome display is similar to mRNA display, but relies on non-covalent interaction in a stalled ribosomal complex to link protein and mRNA during translation. Cell surface display and phage display install the displayed protein on a capsid or membrane protein.

### BRIEF SUMMARY

Various embodiments provide methods, apparatuses, systems, computer program products, and/or the like for performing screening and/or sequencing of molecules using DNA templated molecule synthesis wherein the genotype-phenotype linkage (molecular identity linked with molecular structure/function) is maintained throughout the process. Various embodiments provide methods, apparatuses, systems, computer programs products, and/or the like for performing screening and/or sequencing of kinetic and/or non-equilibrium properties of molecules wherein the genotype-phenotype linkage is maintained throughout the process. Various embodiments provide methods, apparatuses, systems, computer programs products, and/or the like, for observing and/or interrogating molecule behaviors.

Various embodiments provide an imaging instrument and methods for detecting the presence of various molecules in a sample using the imaging instrument. In various embodiments, the molecules are molecules related to biological processes and are detected based on interactions with arrays of clusters of proteins. In an example embodiment, the sample is a biological sample, such as blood serum.

As part of the process, a database of binding properties may be generated. For example, the binding properties corresponding to one or more proteins may be determined. The binding properties may comprise various combinations of equilibrium properties (e.g., binding constant and/or the like), non-equilibrium properties (e.g., association rate, dissociation rate, and/or the like), effects of environmental conditions on binding (e.g., temperature, pH, and/or the like), various other binding functions (e.g., structural changes, catalysis, and/or the like), and/or the like.

As part of the process, a database of enzymatic properties may be generated. For example, the enzymatic properties corresponding to one or more proteins may be determined. For example, the enzymatic properties corresponding to one or more deoxyribozymes or ribozymes may be determined. The enzymatic properties may comprise various combinations of catalytic properties (e.g., catalytic efficiency), specificity, effects of environmental conditions on catalytic events (e.g., temperature, pH, and/or the like), and/or the like.

In various embodiments, a molecular library is generated. For example, a hybridized library may be generated in accordance with mRNA display techniques. For example, an in vitro translated protein may be associated with its coding mRNA via a puromycin linkage. The protein of interest, linked to its coding mRNA, may then be bound to a sequenced and immobilized cDNA transcript of the coding mRNA.

As part of the process, the DNA template for the molecule library will be identified through methods such as next generation sequencing, in various embodiments. While sequencing, the DNA template will be immobilized on a surface that can be viewed by an optical instrument. For example, the surface can be a Miseq flow cell. In various embodiments, a DNA templated molecule library is generated and is immobilized back to the surface through DNA hybridization.

Arrays of clusters of proteins that may be used to identity particular molecules and/or differentiate between multiple molecules may be formed. For example, a focused hybridized library may be prepared, similar to as described above, where the initial DNA library used to generate the hybridized library has been filtered or focused to provide an array of clusters of proteins that may be used to identify particular molecules and/or differentiate between multiple molecules. The filtered or focused hybridized library may also comprise proteins with known affinity towards specific targets (e.g., targets that are expected or known to be present in various samples) to provide a positive control. Samples (e.g., blood serum) may be interacted with the focused hybridized library within a flow cell, and the flow cell may be optically interrogated to determine interactions and bindings that occur between components of the sample and the focused hybridized library. The determined interactions and bindings may then be used to determine the presence or absence of one or more molecules in the sample.

In various embodiments, the binding properties or enzymatic properties for molecules may be determined using flow cytometry. For example, a hybridized library may be generated in accordance with mRNA display techniques. For example, an in vitro translated protein may be associated with its coding mRNA via a puromycin linkage. The protein of interest, linked to its coding mRNA, may then be bound to a sequenced and immobilized cDNA transcript of the coding mRNA. The protein of interest may then be subjected to binding or interaction assays, and the resulting binding interactions may be studied to determine binding properties corresponding to the protein of interest and/or the molecules that interact with the protein. Clusters of proteins of interest may be used to form an array to enable massively parallel screening of binding properties for various molecules. The binding interactions may occur within a flow cell such that the results of the array of proteins of interest being subjected to the binding or interaction assays may be determined by optically interrogating the flow cell (e.g., with one or more laser beams).

In one example, the focused hybridized library may be configured to provide a multiplexed serological assay configured to provide the sample provider's immune history. In another example, a sample may be mixed with one or more molecular switches configured to interact with molecules that may be present in the sample. The mixture may then be provided to the flow cell comprising the focused hybridized library to determine that status of the molecular switches.

In various embodiments, the imaging instrument comprises an optical imaging module and a light source module configured to operate in a multi-laser total internal reflection operational mode, a light-sheet imaging operational mode, a time delay imaging operational mode, or a Fourier ptychography operational mode. The imaging instrument may further comprise a fluid control module capable of precise microliter volume control and a temperature control module with thermoelectric heating/cooling in the 4-97° C. range.

According to an aspect of the present disclosure, a method for determining molecular properties of at least one molecule is provided. In an example embodiment, the method comprises generating a molecule library comprising the at least one molecule; generating a focused library by filtering the molecule library, the focused library comprising the at least one molecule; attaching the focused library to a flow cell of an imaging instrument; controlling a fluid control module of the imaging instrument to incubate the flow cell with a fluid comprising labelled target molecules; operating a light source module and an imaging module of the imaging instrument in a selected operational mode such that the light source module illuminates the flow cell and the imaging module captures light generated by fluorescence by the labelled target molecules within the flow cell; generating imaging data based on the captured light; and analyzing the imaging data to determine the molecular properties of the at least one molecule.

According to another aspect of the present disclosure, an imaging instrument is provided. In an example embodiment, the imaging instrument comprises a flow cell; a fluid control module configured to control a flow of fluid through the flow cell; a light source module configured to illuminate at least a portion of the flow cell; a temperature control module configured to control a temperature within the flow cell; an imaging module configured to capture light emitted from the flow cell; and a controller configured to control operation of the fluid control module, light source module, temperature control module, and imaging module, and to receive signals corresponding to light captured by the imaging module. The controller is configured to operate the imaging instrument in at least one operational mode that enables a sampling rate by the imaging module of less than one minute.

According to still another aspect of the present disclosure a computer program product is provided. In an example embodiment, the computer program product comprises at least one non-transitory computer-readable storage medium storing computer executable code portions, the computer executable code portions comprising computer executable instructions configured to, when executed by a processing element of an apparatus, cause the apparatus to cause performance of generating a molecule library comprising the at least one molecule; generating a focused library by filtering the molecule library, the focused library comprising the at least one molecule; attaching the focused library to a flow cell of an imaging instrument; controlling a fluid control module of the imaging instrument to incubate the flow cell with a fluid comprising labelled target molecules; operating a light source module and an imaging module of the imaging instrument in a selected operational mode such that the light source module illuminates the flow cell and the imaging module captures light generated by fluorescence by the labelled target molecules within the flow cell; generating imaging data based on the captured light; and analyzing the imaging data to determine the molecular properties of the at least one molecule.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 is an overview of a system that can be used to practice embodiments of the present disclosure.

FIG. 2 is an exemplary schematic diagram of a user computing entity, according to one embodiment.

FIG. 3 is a block diagram of an exemplary imaging instrument, according to one embodiment.

FIG. 4 is an exemplary schematic diagram of a controller of an imaging instrument, according to one embodiment.

FIG. 5 is an exemplary schematic diagram of a fluid management system of an imaging instrument, according to an example embodiment.

FIG. 6 is an exemplary schematic diagram of a light source module of an imaging instrument, according to an example embodiment.

FIG. 7 is an exemplary schematic diagram of a temperature management module of an imaging instrument, according to an example embodiment.

FIG. 8 is an exemplary schematic diagram of an imaging module of an imaging instrument, according to an example embodiment.

FIG. 9 is an exemplary schematic diagram of the light source module and the imaging module of the imaging instrument when the imaging instrument is being operated in total internal reflection fluorescence (TIRF) operational mode, according to an example embodiment.

FIG. 10 is an exemplary schematic diagram of the light source module and the imaging module of the imaging instrument when the imaging instrument is being operated in Fourier ptychography (FP) operational mode, according to an example embodiment.

FIG. 11A is an exemplary schematic diagram of the light source module and the imaging module of the imaging instrument when the imaging instrument is being operated in light sheet operational mode, according to an example embodiment.

FIG. 11B is an exemplary schematic diagram of the light source module and the imaging module of the imaging instrument when the imaging instrument is being operated in time delay imaging operational mode, according to an example embodiment.

FIG. 12A is an exemplary flowchart of an example process of operating the imaging instrument, according to an example embodiment.

FIG. 12B illustrates example imaging data and representations of how such imaging data is generated, according to an example embodiment.

FIG. 12C illustrates example imaging data for use in determining, for example, equilibrium properties, according to an example embodiment.

FIG. 12D illustrates example imaging data for use in determining, for example, non-equilibrium properties, according to an example embodiment.

FIG. 12E illustrates an example of capturing imaging data related to non-equilibrium properties of the molecule library and/or focused library, according to an example embodiment.

FIG. 12F illustrates an example of registering results, according to an example embodiment.

FIG. 12G provides an illustration of example intensity linking data, according to an example embodiment.

FIG. 12H provides another illustration of example intensity linking data, according to an example embodiment.

FIG. 13 is an exemplary flowchart of an example process of operating the imaging instrument, according to an example embodiment.

FIG. 14A is an exemplary diagram illustrating an example process of operating the imaging instrument, according to an example embodiment.

FIG. 14B is an exemplary flowchart of an example process of operating the imaging instrument, according to an example embodiment.

FIG. 15A is an exemplary diagram illustrating an example process of operating the imaging instrument, according to an example embodiment.

FIG. 15B is an exemplary flowchart of an example process of operating the imaging instrument, according to an example embodiment.

FIG. 16A is an exemplary diagram illustrating an example process of operating the imaging instrument, according to an example embodiment.

FIG. 16B is an exemplary flowchart of an example process of operating the imaging instrument, according to an example embodiment.

FIG. 16C is an exemplary flowchart of an example process of operating the imaging instrument, according to an example embodiment.

FIG. 17 is an exemplary diagram illustrating an example process of operating the imaging instrument, according to an example embodiment.

FIG. 18 is an exemplary diagram illustrating an example process of identifying, screening, and/or producing biomolecule conjugated nanomaterials, according to an example embodiment.

FIG. 19 is an exemplary flowchart of an example process of operating the imaging instrument, according to an example embodiment.

FIG. 20 is an exemplary diagram illustrating an example process of operating the imaging instrument, according to an example embodiment.

FIG. 21 is an exemplary diagram illustrating an example process of operating the imaging instrument, according to an example embodiment.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. The terms “approximately” and “substantially” are used herein to refer to being within appropriate manufacturing and/or engineering tolerances. Like numbers refer to like elements throughout.

### I. Computer Program Products, Methods, and Computing Entities

Embodiments of the present invention may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and/or flowchart illustrations. Thus, it should be understood that each block of the block diagrams and/or flowchart illustrations may be implemented in the form of a computer program product, an entirely hardware embodiment, a combination of hardware and computer program products, and/or apparatus, systems, computing devices, computing entities, and/or the like, carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### II. Exemplary System Architecture

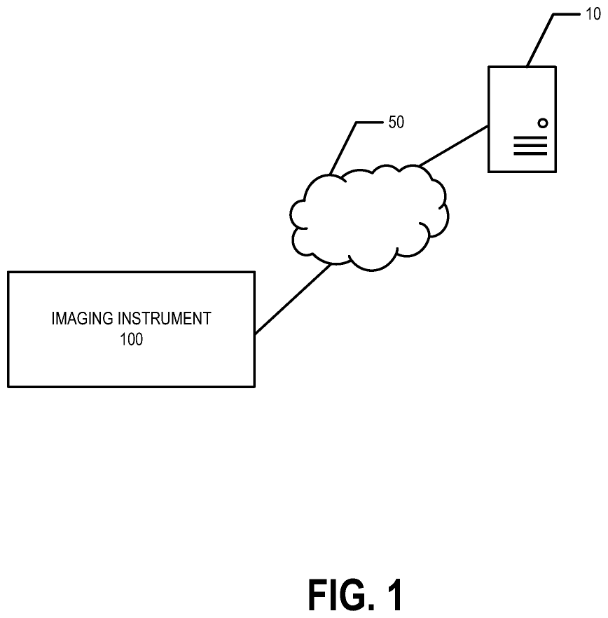

FIG. 1 provides an illustration of an exemplary embodiment of the present disclosure. As shown in FIG. 1, this particular embodiment may include one or more user computing entities 10, an imaging instrument 100, one or more networks 50, and/or the like. Each of these components, entities, devices, systems, and similar words used herein interchangeably may be in direct or indirect communication with, for example, one another over the same or different wired or wireless networks 50. In an example embodiment, a user computing entity 10 is in direct wired or wireless communication with the imaging instrument 100.

While FIG. 1 illustrates the various system entities as separate, standalone entities, the various embodiments are not limited to this particular architecture. In various embodiments, user computing entity 10 and an imaging instrument may be integrated into a single device.

### 1. Exemplary User Computing Entity

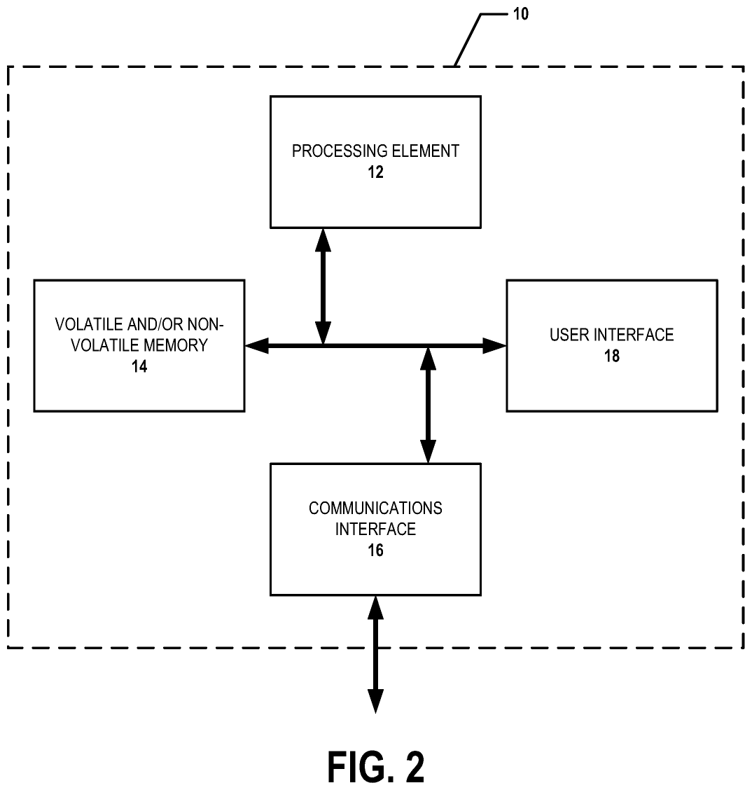

FIG. 2 provides a schematic of a user computing entity 10 according to one embodiment of the present disclosure. In an example embodiment, a user computing entity 10 may be configured to communicate with an imaging instrument 100. For example, the user computing entity 10 may communicate with an imaging instrument 100 to cause the imaging instrument 100 to capture imaging information/data, to receive imaging information/data captured by the imaging instrument 100, and/or the like.

In general, the terms computing entity, computer, entity, device, system, and/or similar words used herein interchangeably may refer to, for example, one or more computers, computing entities, desktops, mobile phones, tablets, phablets, notebooks, laptops, distributed systems, input terminals, servers or server networks, blades, gateways, switches, processing elements, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

In one embodiment, the user computing entity 10 may also include one or more communications interfaces 16 for communicating with various other computing entities, such as by communicating data, content, information, and/or similar terms used herein interchangeably that can be transmitted, received, operated on, processed, displayed, stored, and/or the like.

As shown in FIG. 2, in one embodiment, the user computing entity 10 may include or be in communication with one or more processing elements 12 (also referred to as processors, processing circuitry, and/or similar terms used herein interchangeably) that communicate with other elements within the user computing entity 10 via a bus, for example. As will be understood, the processing element 12 may be embodied in a number of different ways. For example, the processing element 12 may be embodied as one or more complex programmable logic devices (CPLDs), microprocessors, multi-core processors, co-processing entities, application-specific instruction-set processors (ASIPs), microcontrollers, and/or controllers. Further, the processing element 12 may be embodied as one or more other processing elements or circuitry. The term circuitry may refer to an entirely hardware embodiment or a combination of hardware and computer program products. Thus, the processing element 12 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 12 may be configured for a particular use or configured to execute instructions stored in volatile or non-volatile media or otherwise accessible to the processing element 12. As such, whether configured by hardware or computer program products, or by a combination thereof, the processing element 12 may be capable of performing steps or operations according to embodiments of the present invention when configured accordingly.

In one embodiment, the user computing entity 10 may further include or be in communication with memory 14. In an example embodiment, the memory 14 comprises non-volatile media (also referred to as non-volatile storage, memory, memory storage, memory circuitry and/or similar terms used herein interchangeably). In one embodiment, the non-volatile storage or memory may include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the memory 14 may further include or be in communication with volatile media (also referred to as volatile storage, memory, memory storage, memory circuitry and/or similar terms used herein interchangeably). In one embodiment, the volatile storage or memory may also include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 12. Thus, the databases, database instances, database management systems, data, applications, programs, program modules, scripts, source code, object code, byte code, compiled code, interpreted code, machine code, executable instructions, and/or the like may be used to control certain aspects of the operation of the user computing entity 10 with the assistance of the processing element 12 and operating system.

As indicated, in one embodiment, the user computing entity 10 may also include one or more communications interfaces 16 for communicating with various other computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the user computing entity 10 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The user computing entity 10 may also comprise a user interface 18 (that can include a display coupled to a processing element). For example, the user interface 18 may include or be in communication with one or more input elements, such as a keyboard input, a mouse input, a touch screen/display input, motion input, movement input, audio input, pointing device input, joystick input, keypad input, and/or the like. The user computing entity 10 may also include or be in communication with one or more output elements (not shown), such as audio output, video output, screen/display output, motion output, movement output, and/or the like. These input and output elements may include software components, such as a user application, browser, graphical user interface, and/or the like to facilitate interactions with and/or cause display of information/data from the user computing entity 10, as described herein. The user input interface can comprise any of a number of devices or interfaces allowing the user computing entity 10 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the user computing entity 10 and may include a full set of alphabetic keys or set of keys that may be activated to provide a full set of alphanumeric keys. For example, a user may review imaging information/data captured and/or generated by the imaging instrument 100 and/or the result of analyzing and/or processing imaging information/data via the user interface 18.

As will be appreciated, one or more of the components of the user computing entity 10 may be located remotely from other components of the user computing entity 10, such as in a distributed system. Furthermore, one or more of these components may be combined with additional components to perform various functions described herein, and these additional components may also be included in the user computing entity 10. Thus, the user computing entity 10 can be adapted to accommodate a variety of needs and circumstances. As will be recognized, these architectures and descriptions are provided for exemplary purposes only and are not limiting to the various embodiments.

### 2. Exemplary Imaging Instrument

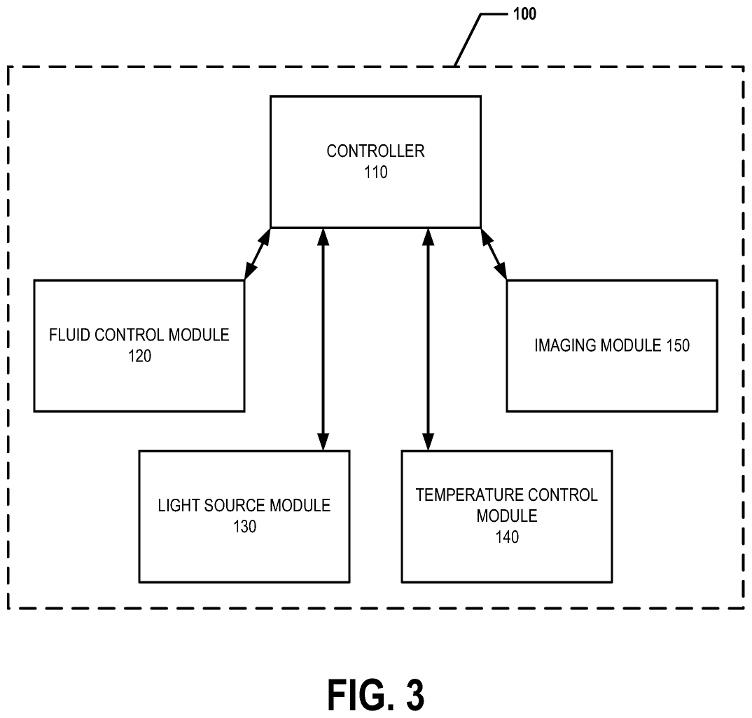

In various embodiments, an imaging instrument is configured for capturing and/or generating imaging information/data indicating reactions, interactions, and/or the like (or the lack thereof) that occur between molecules, reagents, and/or the like within a flow cell of the imaging instrument. As shown in FIG. 3, an example embodiment of the imaging instrument 100 comprises a controller 110, a fluid control module 120, a light source module 130, a temperature control module 140, and an imaging module 150. The controller 110 is configured to operate and/or control operation of the fluid control module 120, the light source module 130, the temperature control module 140, and the imaging module 150 in one or more operational modes. In various embodiments, the one or more operational modes include a total internal reflection fluorescence (TIRF) operational mode, Fourier ptychography (FP) operational mode, light sheet imaging operational mode, time delay imaging (TDI) operational mode, and/or the like. In various embodiments, at least on operational mode enables the capturing and/or generating of imaging information/data corresponding to equilibrium (e.g., binding constant) and non-equilibrium (e.g., association and dissociation rate) properties and multiple functions (e.g., binding, structure switching and catalysis) for multiple molecules simultaneously and under various conditions (i.e. temperature and pH).

### A. Exemplary Controller

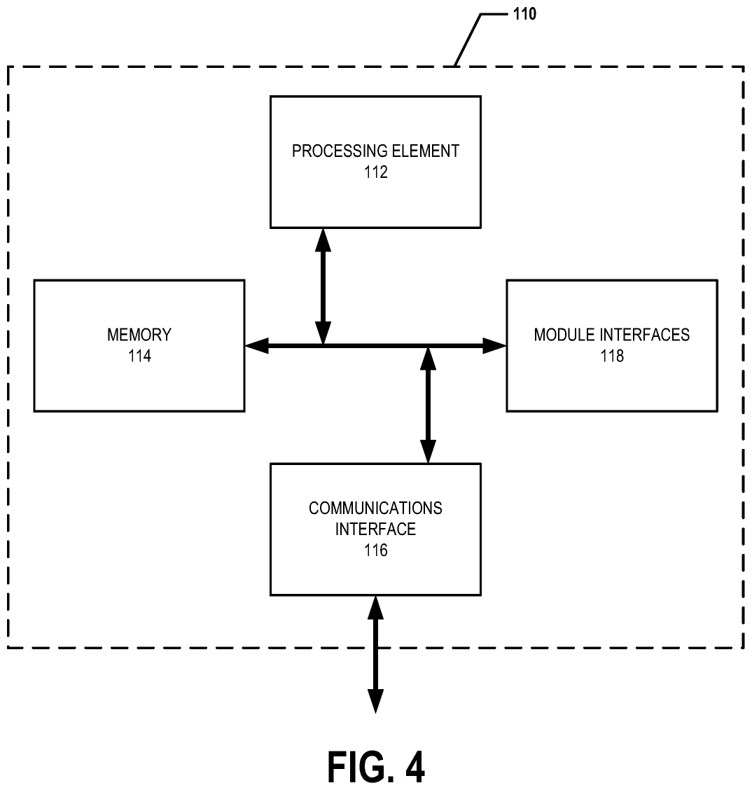

In various embodiments, the controller 110 may be a computing entity and/or a component of a computing entity (e.g., microprocessor, processor, memory, field-programmable gate array (FPGA), and/or other component comprising an integrated circuit). FIG. 4 provides a block diagram of an example controller 110, according to an example embodiment. In the illustrated embodiment, the controller 110 comprises at least one processing element 112, memory 114, one or more communications interfaces 116 configured to communicate via one or more networks, and one or more module interfaces 118.

As shown in FIG. 4, in one embodiment, the controller 110 may include or be in communication with one or more processing elements 112 (also referred to as processors, processing circuitry, and/or similar terms used herein interchangeably) that communicate with other elements within the controller 110 via a bus, for example. As will be understood, the processing element 112 may be embodied in a number of different ways. For example, the processing element 112 may be embodied as one or more CPLDs, microprocessors, multi-core processors, co-processing entities, ASIPs, microcontrollers, and/or controllers. Further, the processing element 112 may be embodied as one or more other processing elements or circuitry. The term circuitry may refer to an entirely hardware embodiment or a combination of hardware and computer program products. Thus, the processing element 112 may be embodied as integrated circuits, ASICs, FPGAs, PLAs, hardware accelerators, other circuitry, and/or the like. As will therefore be understood, the processing element 112 may be configured for a particular use or configured to execute instructions stored in volatile or non-volatile media or otherwise accessible to the processing element 112. As such, whether configured by hardware or computer program products, or by a combination thereof, the processing element 112 may be capable of performing steps or operations according to embodiments of the present invention when configured accordingly.

In the illustrated embodiment, the controller 110 further includes or is in communication with memory 114. In an example embodiment, the memory 114 comprises non-volatile media (also referred to as non-volatile storage, memory, memory storage, memory circuitry and/or similar terms used herein interchangeably). In one embodiment, the non-volatile storage or memory may include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the memory 114 further includes or is in communication with volatile media (also referred to as volatile storage, memory, memory storage, memory circuitry and/or similar terms used herein interchangeably). In one embodiment, the volatile storage or memory may also include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 12. Thus, the databases, database instances, database management systems, data, applications, programs, program modules, scripts, source code, object code, byte code, compiled code, interpreted code, machine code, executable instructions, and/or the like may be used to control certain aspects of the operation of the controller 110 (and the imaging instrument 100) with the assistance of the processing element 112.

As indicated, in one embodiment, the controller 110 may also include one or more communications interfaces 116 for communicating with various other computing entities (e.g., a user computing entity 10), such as by communicating imaging information/data, data, content, information, and/or similar terms used herein interchangeably that can be transmitted, received, operated on, processed, displayed, stored, and/or the like. Such communication may be executed using a wired data transmission protocol, such as FDDI, DSL, Ethernet, ATM, frame relay, DOCSIS, or any other wired transmission protocol. Similarly, the user computing entity 10 may be configured to communicate via wireless external communication networks using any of a variety of protocols, such as GPRS, UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR protocols, NFC protocols, Wibree, Bluetooth protocols, wireless USB protocols, and/or any other wireless protocol.

The controller 110 also comprises one or more module interfaces 118. In various embodiments, the module interfaces 118 are configured to enable the controller 110 to communicate with, control, and/or receive information/data from one or more of the fluid control module 120, light source module 130, temperature control module 140, and imaging module 150. For example, the module interfaces 118 may include various drivers, analog/digital converters, and/or the like. For example, the module interfaces 118 may comprise one or more laser drivers for operating one or more lasers of the light source module 130.

In an example embodiment, the controller 110 is also in communication with a user interface (not shown). For example, the user interface may include or be in communication with one or more input elements, such as a keyboard input, a mouse input, a touch screen/display input, motion input, movement input, audio input, pointing device input, joystick input, keypad input, buttons, knobs, and/or the like. The controller 110 may also include or be in communication with one or more output elements (not shown), such as audio output, video output, screen/display output, motion output, movement output, dials, and/or the like. These input and output elements may include software components such as a user application, graphical user interface, and/or the like to facilitate interactions with and/or cause display of information/data from the controller 110, as described herein. The user input interface can comprise any of a number of devices or interfaces allowing the controller 110 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the imaging instrument 100 and may include a full set of alphabetic keys or set of keys that may be activated to provide a full set of alphanumeric keys. For example, a user may cause imaging information/data to be captured using a particular operational mode, review imaging information/data captured and/or generated by the imaging instrument 100 and/or the result of analyzing and/or processing imaging information/data, and/or the like via the user interface.

### B. Exemplary Fluid Control Module

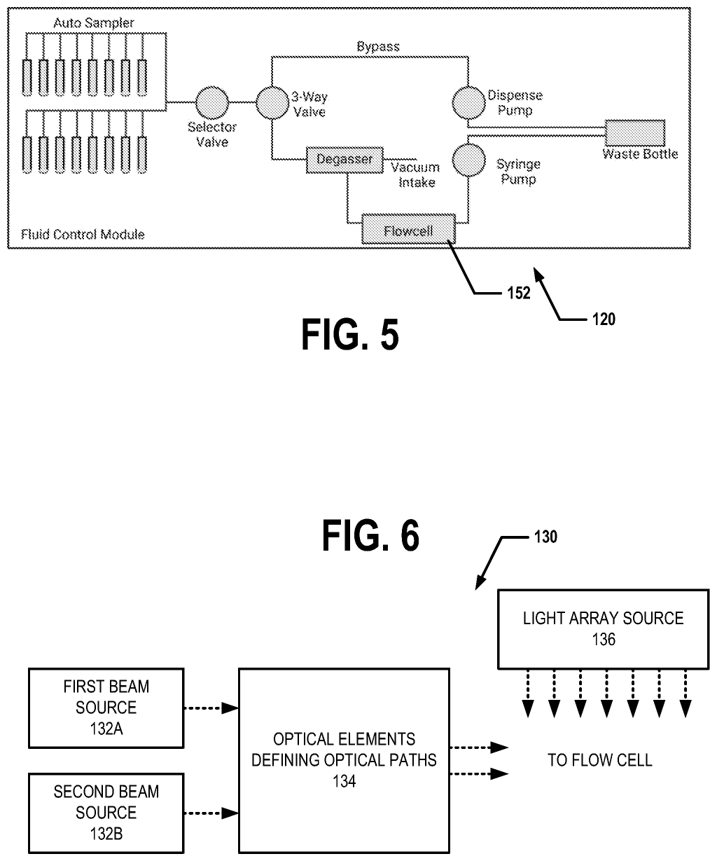

FIG. 5 illustrates a fluid control module 120 of an example embodiment. In various embodiments, the fluid control module 120 is able to prime, purge, and/or clean either with or without involving the sample flow cell. In an example embodiment, the fluid control module 120 is capable of injecting fluids into the flow cell 152 with flow rate between 2 μL/min to 1000 μL/min with automated sample selection. The fluid control module 120 is capable of working with different types of reagents including organic solvents, aqueous solutions, complex biofluids, etc. Each flow cell 152 has respective independent inlets and outlets, allowing the flow to run in and out in the same direction. For example, in FIG. 5, the flow of fluids into, through, and out of the flow cell 152 is from left to right.

### C. Exemplary Light Source Module

In various embodiments, the light source module 130 is configured to provide light to illuminate the flow cell 152. For example, the light source module 130 may comprise one or more lasers (e.g., two or more lasers having different characteristic wavelengths), incoherent light sources (e.g., an array of light emitting diodes (LEDs)), and/or other light sources. In various embodiments, the light source module 130 also comprises various optical elements configured to condition the light being provided to the flow cell 152. For example, conditioning the light being provided to the flow cell 152 includes providing the light at a desired position, tuning the wavelength of the light, adjusting the beam width, focusing the light at a desired position, providing an optical path from the light source to the flow cell 152, adjusting the spatial profile of the light, and/or the like.

FIG. 6 provides a schematic view of a light source module of an example embodiment. For example, the light source module 130 comprises a first beam source 132A, a second beam source 132B, optical elements 134 for conditioning beams generated by the first and/or second beam sources 132A,B, and a light array source 136. In an example embodiment, the first and second beam sources 132A,B are lasers that have different characteristic wavelengths. In an example embodiment, the light array source 136 is an array of incoherent light sources such as LEDS.

In various embodiments, the light source module 130 may include various illumination sources. For example, multiple laser lines, xenon lamp, halogen lamp, deuterium lamp or LED lights and arrays may be used as illumination sources in various embodiments. The illumination light of chosen wavelength and spatial profile is directed to the imaging module by configuring light path with mirrors, dichroic optical elements (e.g., beam splitters, filters, and/or the like), lenses, and/or gratings. The light path configuration is controlled by a software and implemented in motor-driven optics mounts (e.g., controlled by respective module interfaces 118). Various operational modes correspond to various illumination modes, such as TIRF, wide field, confocal, darkfield, phase contrast, laser scanning, can be selected and implemented automatically, in various embodiments.

### D. Exemplary Temperature Control Module

In various embodiments, the temperature control module 140 is configured to control the temperature within the flow cell 152 and/or of fluids in various locations within the fluid control module 120. A schematic diagram of a temperature control module 140 of an example embodiment is shown in FIG. 7. In various embodiments, the temperature control module 140 comprises a thermal couple (TC) or other temperature sensor configured to determine and/or measure the temperature within the flow cell 152 and/or fluid within the flow cell 152. In various embodiments, the temperature control module 140 comprises a heat exchanger and a thermal electric controller (TEC). In the temperature control module 140, the TC, TEC, and the heat exchanger are in communication with the controller 110 (e.g., via one or more appropriate module interfaces 118). The TEC is used to heat up the flow cell 152 on the sample stage. The flow cell 152 can then be cooled by liquid circulating with a heat exchanger. The temperature in the flow cell can be managed by heating and cooling in the 4-97° C. range.

### E. Exemplary Imaging Module

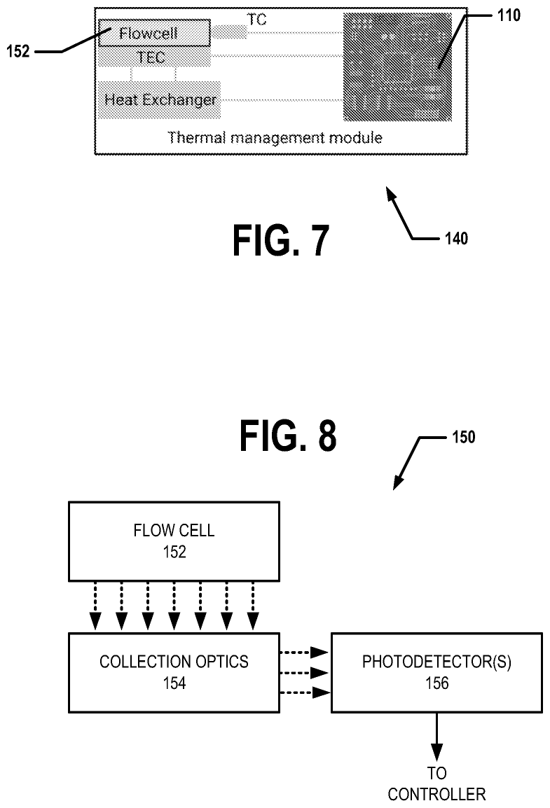

FIG. 8 provides a block diagram of an imaging module 150, according to an example embodiment. In general, the imaging module 150 comprises collection optics 154 configured to collect light emitted from within the flow cell 152 and/or scattered out of the flow cell 152 and provide the collected light to one or more photodetectors 156 of the imaging module. In various embodiments, the photodetectors 156 comprise cameras, charge-coupled devices (CCDs), photodiodes, complementary metal-oxide-semiconductor (CMOS) sensors, photomultiplier tubes, and/or other photodetectors. The photodetectors 156 are in communication with the controller 110 (e.g., via appropriate module interfaces 118).

In the imaging module 150, light reflected or emitted by samples in the flow cell 152 is used to generate an electric signal via photodetector. Two configurations are possible, in an example embodiment, with each configuration corresponding to at least one operational mode of the imaging instrument 100. In a first configuration, a high numerical aperture (NA), infinity-corrected objective is used with a tube lens to capture images on the photodetector 156. In a second configuration, an array of low NA lenses is used to record images on an array of photodetectors to achieve large field-of-view. The types of photodetectors include 1D, time delay imaging (TDI), or 2D CCD and/or CMOS sensors with either passive or active cooling. An infrared laser and a CCD camera are included along with a polarizing beam splitter (PBS) and a tube lens (if infinite space objective is used) for auto focusing, in an example embodiment.

### F. Exemplary Operational Modes

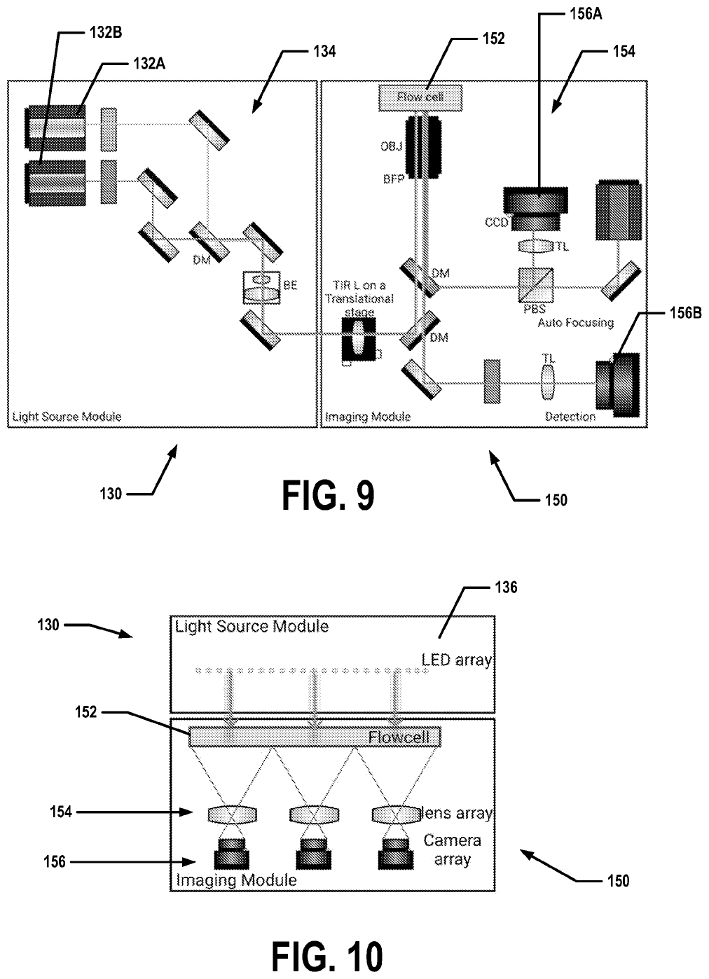

FIG. 9 illustrates an example configuration for the light source module 130 and the imaging module 150 for use in the TIRF operational mode, according to an example embodiment. For the TIRF operational mode, multiple laser lines generated by first and second beam sources 132A,B with good beam quality (e.g., beam propagation ratio or beam quality factor M2 value <1.2) and high output power (e.g., >30 mW) is used in the light source module 130. Different laser lines are well aligned with each other by using reflecting mirrors, possibly including one or more deformable mirrors (DM). A total internal reflection (TIR) focusing lens is used to focus light on the back focal plane (BFP) of the objective (OBJ) after the beams have been expanded through a beam expander (BE). In the sample imaging module 150, an infinite objective (OBJ) with high numerical aperture (e.g., >1.45 NA) and a sample stage compatible with sequencing flow cell 152 and standard microscope slides (e.g., ˜ 75 mm×25 mm) are mounted on the translation stages, which can be controlled either using a joystick controller or as programmed in the controller 110. The emission filters are selected with increased blocking for greater attenuation of TIRF lasers (OD8). An infinity corrected tube lens (TL) is used to focus light after passing through the infinite space where the autofocus module and temperature control module 140 are implemented. An actively cooled high sensitivity CMOS or CCD camera is implemented as the photodetector 156. In the auto focusing module, a testing laser and a CCD camera is included along with polarizing beam splitter (PBS) and a tube lens (TL). By using the TIRF mode microscope system, the excitation and detection of fluorescent signals are restricted to a thin layer of the specimen. The signal to noise ratio is improved by elimination of the background fluorescence outside of the thin layer. Thus, the spatial resolution of the features on the surface are improved.

Example configurations of the light source module 130 and the imaging module 150 for use in the Fourier ptychography (FP) operational mode are shown in FIG. 10. In an example embodiment, the FP operational mode is configured to enable imaging of the entire flow cell 152 on a sub-minute time scale such that non-equilibrium properties of displayed molecules may be observed and/or determined. For example, in various embodiments, the FP operational mode is configured to enable imaging of the flow cell 152 in less than one minute, less than thirty seconds, less than ten seconds, less than five seconds, approximately one second or less, and/or the like. In the illustrated embodiment, an LED array is implemented as the light array source 136 in the imaging module for illumination of the interior flow cell 152. The light path is directed using reflecting mirrors. In the illustrated configuration of the imaging module 150, a lens array is implemented as the collection optics 154. A sample stage compatible with sequencing flow cell 152 and standard microscope slides (e.g., ˜ 75 mm×25 mm) are mounted on the translation stages, which can be controlled either using a joystick controller or as programmed in the controller 110. The photodetectors 156 comprise an array of light detectors that are used to form images and these images are stitched together (e.g., by the controller 110) to produce a wide-filed, high resolution complex sample image.

Conventional microscope systems sacrifice field-of-view for sensitivity, which limits conventional imaging systems' abilities to capture fast molecular events. Due to these limitations, conventional imaging systems are not capable of characterizing the non-equilibrium molecular functions which happen on very short time scales (e.g., less than a minute, less than thirty seconds, less than ten seconds, less than five seconds, approximately one second or less, and/or the like). In the FP operational mode, each LED in the programmable LED array illuminates a different angle or different portion of the flow cell 152 and produces a shift in the spatial frequency. Each lens in the lens array is responsible for sampling a distinct (spatial) range of light coming from the sample. For example, each lens in the lens array is responsible for sampling light coming from the sample located in a particular region of the flow cell 152. Low resolution images (e.g., low-pass band-limited images) are then formed on the array of photodetectors 156. An image process pipeline is then used (e.g., by the controller 110 and/or user computing entity 10) to stitch together the band limited images in Fourier space and produce a wide-field, high resolution complex sample image. The estimation and correction of known and/or unknown aberrations are facilitated by the image-reconstruction algorithms, in various embodiments. The effective numerical aperture is influenced by both the illumination and the synthesized object-space. Thus, both the increase in the number of LEDs and lenses will lead to the increase of signal-to-noise ratio and resolution. Through increasing the number of LEDs in the light array source 136, lenses in the lens array of the collection optics 154, and cameras in the array of photodetectors 156, the field-of-view may be increased without compromising the high resolution and sensitivity.

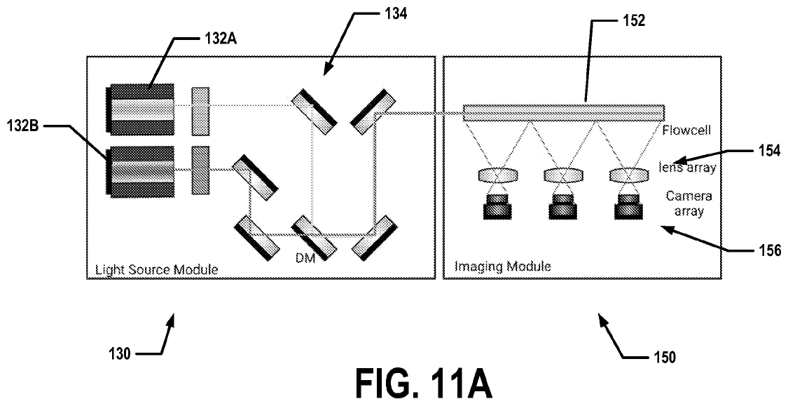

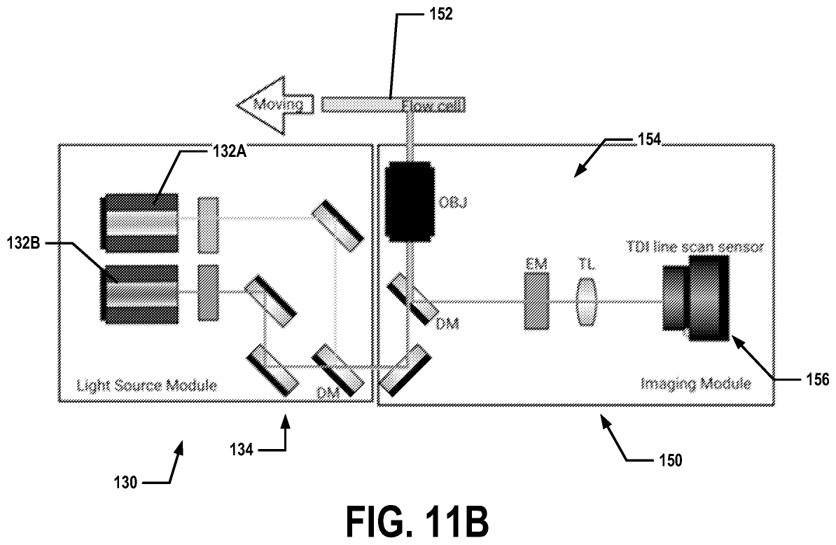

FIG. 11A illustrates example configurations of the light source module 130 and the imaging module 150 for use in the light sheet operational mode. In an example embodiment, the light sheet operational mode is configured to enable imaging of the entire flow cell 152 on a sub-minute time scale such that non-equilibrium properties of displayed molecules may be observed and/or determined. For example, in various embodiments, the light sheet operational mode is configured to enable imaging of the flow cell 152 in less than one minute, less than thirty seconds, less than ten seconds, less than five seconds, approximately one second or less, and/or the like. In light-sheet imaging mode, the laser lines generated by the first and second beam sources 132A,B in the light source module 130 are directed to the flow cell 152 with a light path perpendicular to the direction of observation. Mirrors and dichroic filters are used as the optical elements 134 defining the light path to condition the light path. In the focal region, a thin light-sheet will be created and is used to excite the fluorophores only in a thin layer of the sample. Different types of light sheets may be formed, such as planar light sheets, scanned light sheets, multi-photon light sheets, and scanned Bessel beam and optical lattice light sheets. The emitted fluorescent light from the light sheet will be collected by a lens array of the collection optics 154 and projected on the camera array of the photodetectors 156. Low resolution images formed on the light detectors will then be stitched together through an image processing pipeline and produce a high-resolution complex sample image. The light sheet fluorescent microscope mode has a good optical sectioning capability with an intermediate to high optical resolution. Similar to the FP operational mode, the light sheet operational mode enables the imaging instrument 100 to be used to explore and/or characterize non-equilibrium characteristics of molecules.

FIG. 11B illustrates example configurations of the light source module 130 and the imaging module 150 for use in the time delay imaging operational mode. The time delay imaging operational mode is configured to enable scanning the entire sequencing flow cell 152 on a sub-minute time scale such that non-equilibrium properties of displayed molecules may be observed and/or determined. In the time delay imaging operational mode, the laser lines generated by the first and/or second beam sources 132A,B (e.g., a first and second laser) in the light source module 130 are directed to the flow cell 152. For example, mirrors and dichroic filters (e.g., dichroic mirrors (DM)) are used as the optical elements 134 to define the light path and to condition the light generated by the first and second beam sources 132A,B along the light path, in various embodiments. The light path can be configured to work for either epi florescence imaging or total internal reflection florescence imaging conditions. A sample stage compatible with sequencing flow cell and standard microscope slides (e.g., ˜ 75 mm×25 mm) are mounted on the translation stages, which can be controlled as programmed by the controller 110. The translation stage motion is programmed to be synchronized with the exposures of the time delay and integration line scan sensor (e.g., photodetector(s) 156). The emission light from the sample plane will pass through a combination of lenses which include an objective, a tube lens, emission filters, and reflective mirrors. For example, in various embodiments, the collection optics 154 used in the time delay imaging operational mode include one or more of an objective, a tube lens, emission filters, and/or reflective mirrors. The emission light is then collected by the TDI line scan sensor (e.g., photodetector(s) 156) and provided to the controller 110 for processing, storage, and/or transmission. The line information captured by the TDI line scan sensor (e.g., photodetector(s) 156) is copied line by line synchronously with the translation stage movement and exposed with the same image information. The multiple exposure of the same moving sample is accumulated and increases the integration time available to collect the emission light.

### III. Exemplary System Operation

Example embodiments of the present disclosure provide for operations of an imaging instrument, such as an imaging instrument 100 comprising a controller 110, a fluid control module 120, a light source module 130, a temperature control module 140, and an imaging module 150, as described above. In general, example embodiments of the present disclosure provide for high-throughput screening of a molecule library, and characterization of both equilibrium and non-equilibrium properties of molecules of the molecule library while maintaining a genotype-phenotype linkage. In various example embodiments, an imaging instrument 100 may be operated to image a flow cell 152 and detect the various molecules (e.g., and their fluorescence) attached and/or displayed on the flow cell 152. For example, various molecules may be displayed using display techniques (e.g., mRNA display, ribosomal display, cell surface display, phage display, or other DNA templated molecule synthesis methods) on a flow cell 152. Throughout the screening/filtering of the library and the determination of the molecules' equilibrium and non-equilibrium properties, the physical genotype-phenotype linkage is maintained. In various example embodiments, a database of binding properties may be generated based on observation of various molecules displayed on a flow cell 152 using an imaging instrument 100. The database of binding properties may maintain a genotype-phenotype linkage; for example, the database may store sequencing data (e.g., nucleotide sequences) associated with each molecule's binding properties. Example embodiments of the present disclosure provide for operations of directed evolution for producing various molecules, such as oligonucleotides, riboswitches, catalytic enzymes, and/or affimers, with desired properties (e.g., binding properties).

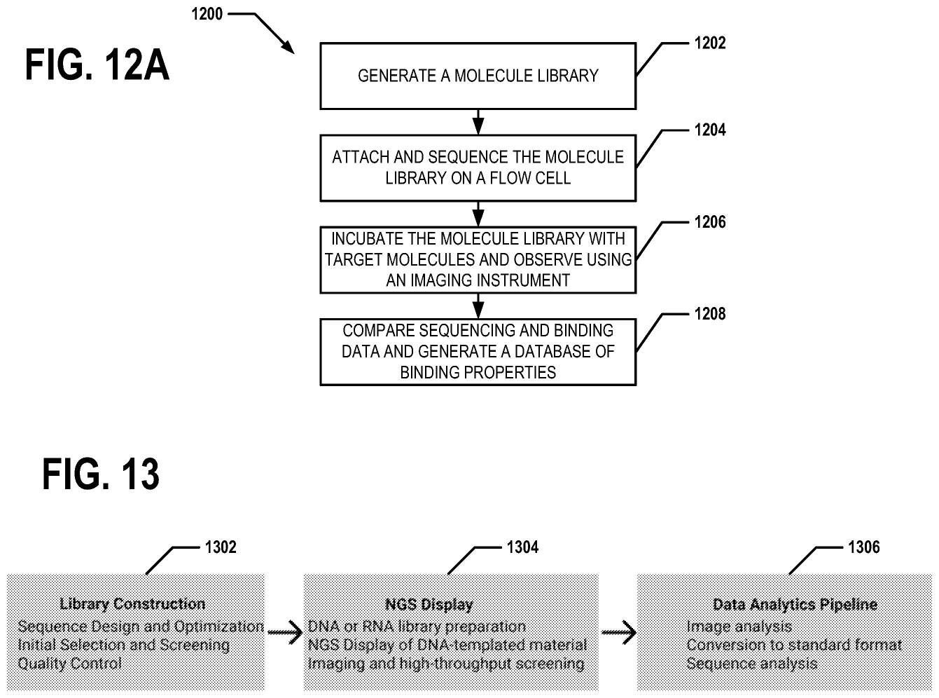

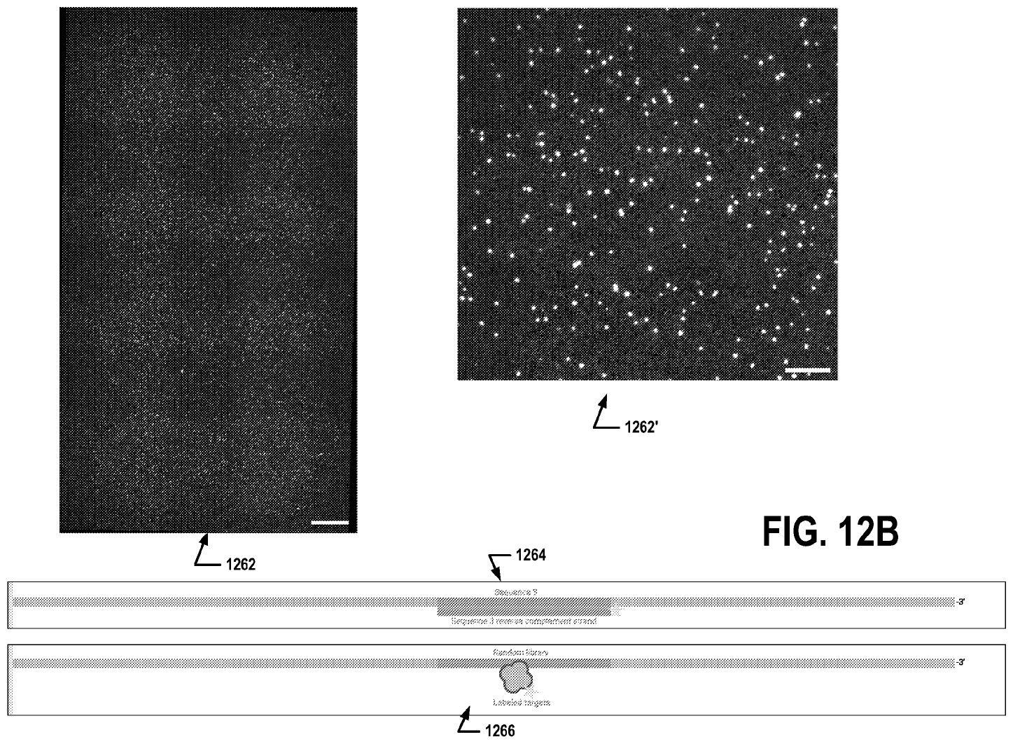

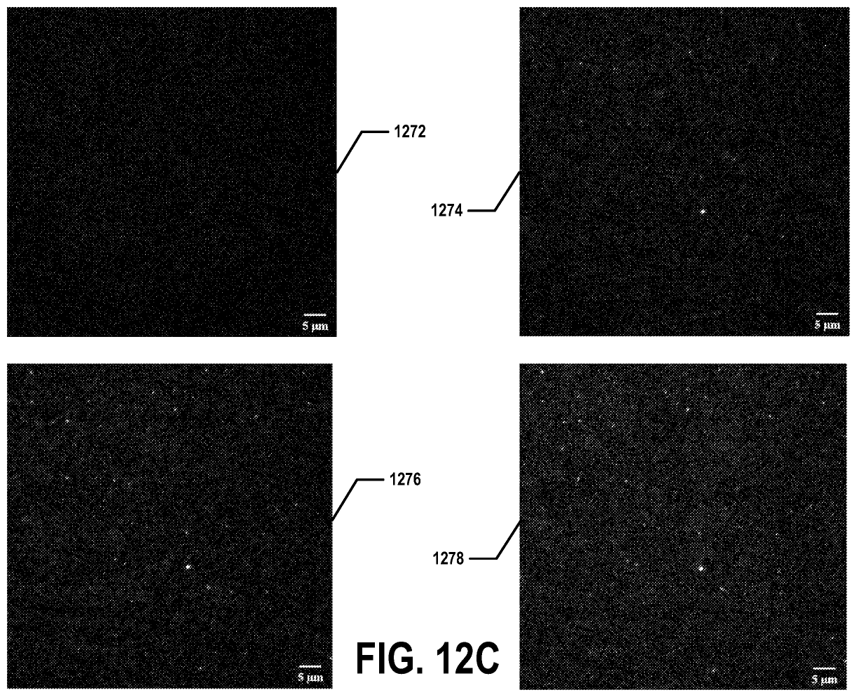

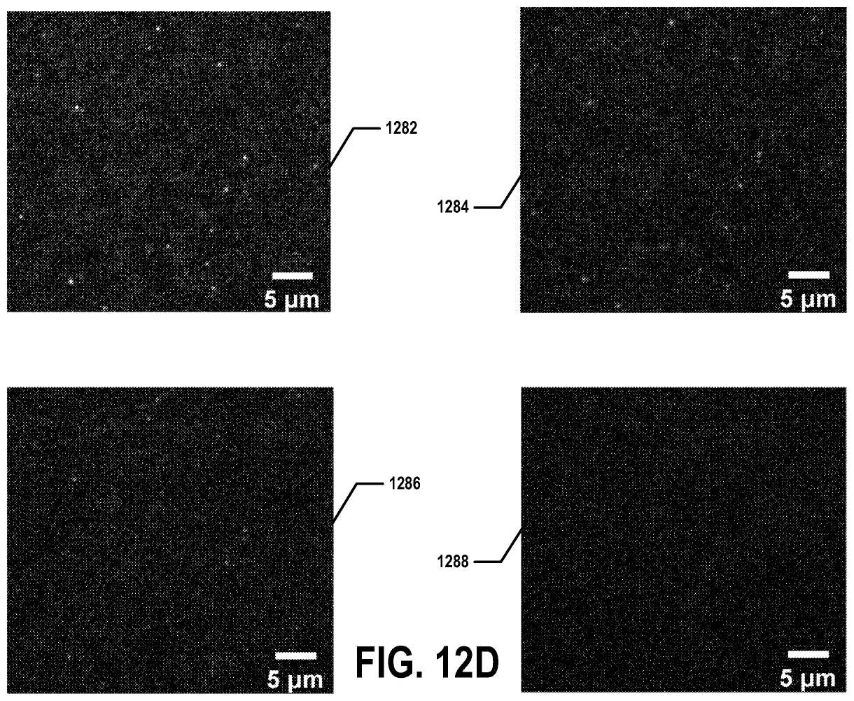

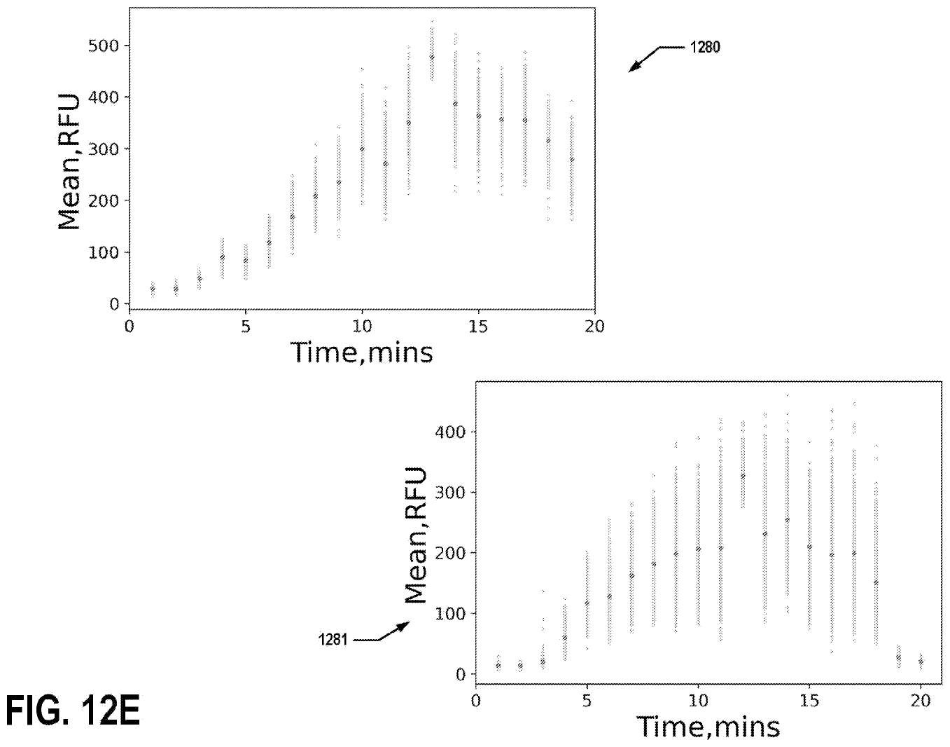

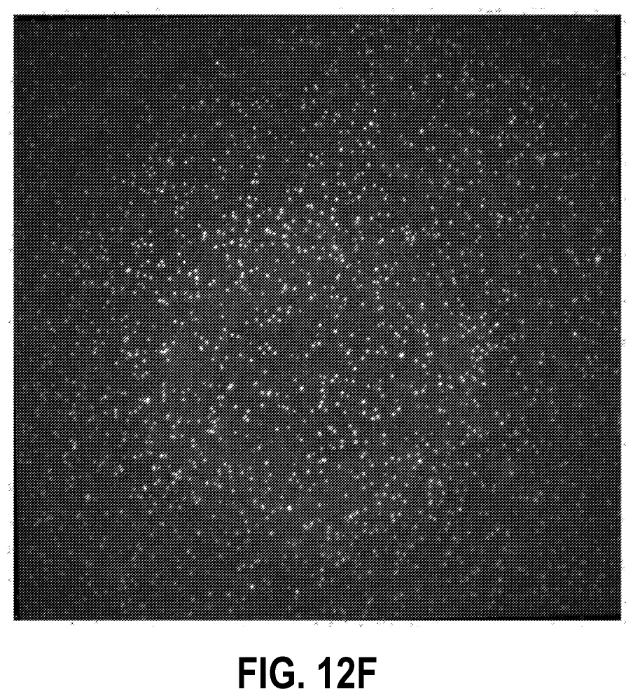

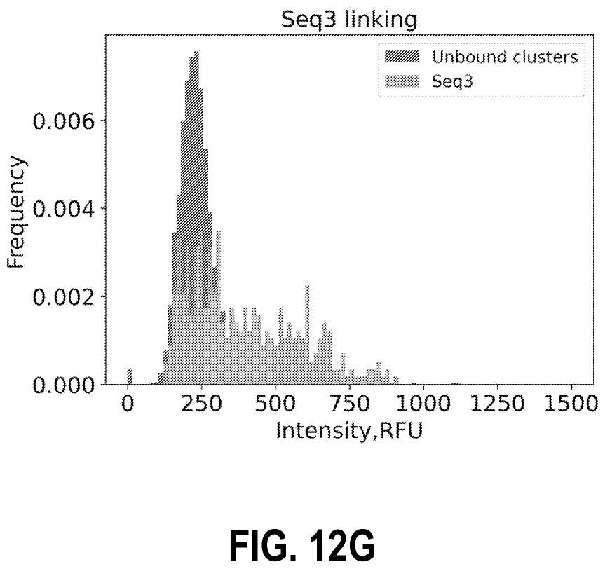

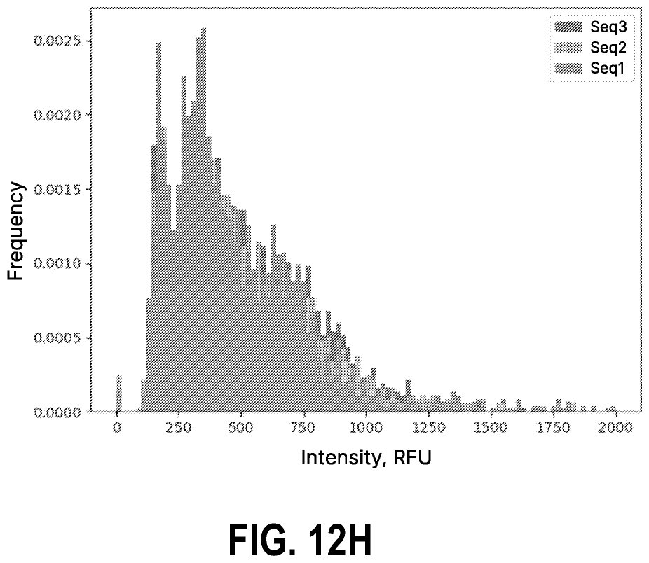

FIG. 12A provides a flowchart of an operation of an imaging instrument 100 according to one embodiment of the present disclosure. For example, the imaging instrument may be operated as part of generating a database of information regarding equilibrium and non-equilibrium properties of molecules, down selecting and/or filtering a library of molecules, and/or the like, in various embodiments. In the illustrated embodiment, an example operation begins at step/operation 1202, which comprises generating a molecule library. In various example embodiments, the user computing entity 10 comprises means, such as processing element 12, memory 14, communications interface 16, user interface 18, and/or the like, for generating a molecule library. For example, the user computing entity 10 comprises a memory 14 storing instructions for generating a molecule library (e.g., from an existing molecule library), producing a molecule library, data on existing molecule libraries, data on desired molecular properties in the constructed library, and/or the like, and a processing element 12 for performing stored instructions. For example, the user computing entity 10 comprises a communications interface 16 configured to communicate (e.g., via one or more networks 50) with other user computing entities 10 and/or other databases to obtain data for generating a molecule library, transmit instructions/requests for generating a molecule library, and/or the like. Specifically, the user computing entity may comprise a communications interface 16 configured to communicate with a controller of a device configured to generate a molecule library. For example, the user computing entity comprises a user interface 18 for receiving user input for generating a molecule library and outputting data relating to generating a molecule library to a user. Various steps for generating the molecule library may be performed by, for example, a human lab technician, in various embodiments.

The molecule library may be one of a DNA library, a complementary DNA (cDNA) library, a messenger RNA (mRNA) library, a protein library, a DNA templated molecule, and/or any molecule library storing molecules, nucleotide sequences, peptides, and/or proteins of interest. The molecule library may have a size in the range of 10¹⁴⁻²³ molecules. In various example embodiments, the molecule library may comprise random molecules, molecules with varying nucleotide sequences, and/or molecules with varying characteristics. In various embodiments, the molecule library comprises organic and/or non-organic molecules.

In various example embodiments, step/operation 1202 of generating a molecule library may comprise at least a portion of step/operation 1302 of FIG. 13, which comprises library construction. In other words, generating a molecule library may comprise constructing a molecule library. As illustrated in FIG. 13, molecule library construction may comprise sequence design and optimization. For example, a nucleotide sequence of interest or a desired nucleotide sequence may be designed, and a molecule library comprising a molecule including the nucleotide sequence of interest of desired nucleotide sequence may be constructed and/or synthesized. Furthermore, the nucleotide sequence of interest/desired nucleotide sequence and/or molecules including the nucleotide sequence of interest/desired nucleotide sequence may be optimized, in an example embodiment. In various example embodiments, optimization of the nucleotide sequence of interest/desired nucleotide sequence comprises removing introns, eliminating stop codons or termination codons, selecting preferred and/or specific codons, and/or the like. Molecule library construction may further comprise initial selection and screening. For example, in a general example embodiment, the number of molecules in the constructed molecule library may be reduced, such as by removing undesired molecules. In an example embodiment, molecular screening may be performed to reduce the number of molecules in the constructed molecule library. For example, the molecular screening may include filtering based on positive selection (e.g., a property that a molecule does possess) and/or negative selection (e.g., a property that a molecule does not possess). Molecule library construction additionally comprises quality control. The constructed molecule library may be analyzed to determine whether the molecule library, and/or molecules of the molecule library satisfy certain quality requirements. For example, quality requirements may include absence of stop codons, presence of the nucleotide sequence of interest without missing (e.g., truncated from the 3′-end or the 5′-end) nucleotides, a threshold diversity amount or value, a threshold number of copies for each molecule, and/or the like.

The result of filtering the molecule library (e.g., based on positive and/or negative selection) is a focused library.

Returning to FIG. 12A, step/operation 1204 may follow step/operation 1202. Step/operation 1204 comprises attaching and sequencing the (filtered and/or down-selected) molecule library on a flow cell 152. In various example embodiments, the user computing entity 10 comprises means, such as processing element 12, memory 14, communications interface 16, user interface 18, and/or the like, for attaching the molecule library to a flow cell 152. For example, the user computing entity 10 comprises a memory 14 storing instructions for attaching the molecule library to the flow cell 152 and a communications interface 16 configured to communicate with a controller of a device configured to attach the molecule library to the flow cell 152. Various steps for attaching the molecule library to the flow cell 152 may be performed by, for example, a human lab technician and/or a device configured to attach a molecule library to a flow cell 152, in various embodiments. In various example embodiments, the flow cell 152 may comprise one or more sections where molecules of the molecule library may be attached. Each section of the flow cell 152 may contain different attached molecules and/or molecules with different nucleotide sequences, properties, characteristics, and/or the like.

In various example embodiments, step/operation 1204 comprises preparing the flow cell 152, such as by determining a fluidic flow rate, including polymerases, deoxynucleotide triphosphates (dNTPs), and/or buffers in a fluid flowing through the flow cell 152, immobilizing oligonucleotides on a surface of the flow cell 152, and/or the like. In an example embodiment, the flow cell 152 may be an Illumina® MiSeq flow cell. In an example embodiment, attaching the molecular library to the flow cell 152 comprises performing sequencing cluster generation. For example, molecules of the molecule library, are hybridized to immobilized oligonucleotides on a surface of the flow cell 152, where they are copied (e.g., by 3′ extension). In an example embodiment, copies of the DNA templates and/or molecules of the molecule library are generated from the immobilized oligonucleotides using high-fidelity DNA polymerase. These copies may then loop over to hybridize with adjacent immobilized oligonucleotides, forming bridge structures. DNA polymerase may then form copies of the bridge structures originating from the adjacent immobilized oligonucleotides, resulting in double-stranded molecule bridges. Then, each double-stranded bridge may be denatured to form two single-stranded molecules attached to the surface of the flow cell 152. Using sequencing cluster generation, molecule clusters with more than one copy of each molecule of the molecular library may be formed and attached to the flow cell 152, allowing for high data density.

In various example embodiments, step/operation 1204 may comprise at least a portion of step/operation 1304 illustrated in FIG. 13. Step/operation 1304 comprises next-generation sequencing (NGS) display or displaying of various molecules on molecules of the molecule library. For example, NGS display may comprise preparing a DNA or RNA library on a flow cell 152, such as by attaching molecules of the DNA or RNA library to the flow cell 152. The RNA library may be an mRNA library. Then, DNA-templated material may be displayed. For example, materials, or molecules, may be configured and/or synthesized to have complementary nucleotide sequences to molecules of the DNA or RNA library attached to the flow cell 152, and may attach to respective complementary molecules. Various molecular display techniques may be used in NGS display of DNA-templated material and based at least in part on molecules of the molecule library. For example, mRNA display techniques may be used for an mRNA library attached to the flow cell 152, where templated material may attach to mRNA molecules via covalent chemical bonds.

Returning to process 1200 in FIG. 12A, step/operation 1206 may follow step/operation 1204. Step/operation 1206 comprises incubating the molecule library and/or focused library and the flow cell 152 with target molecules and observing the flow cell 152 using the imaging instrument 100. In various example embodiments, the user computing entity 10 comprises means, such as processing element 12, memory 14, communications interface 16, user interface 18, and/or the like, for incubating the molecule library and/or focused library with target molecules and observing the flow cell 152 using the imaging instrument 100. For example, the user computing entity 10 comprises a communications interface 16 configured to communicate with controller 110 of the imaging instrument 100 to cause the flow cell 152 to be incubated at a particular incubation temperature, to introduce target molecules to the flow cell, to cause the capture of imaging information/data of the molecule library and/or focused library, to receive and/or obtain imaging information/data captured by the imaging instrument 100, and/or the like. For example, the user computing entity 10 comprises a processing element 12 (e.g., processor) for selecting an operational mode (e.g., TIRF mode, FP mode, light sheet imaging mode, and/or another operational mode) of the imaging instrument 100 that may capture optimal imaging information/data for a particular operation of the imaging instrument 100 based on imaging properties of each operational mode, and a communications interface 16 for communicating with the controller 110 of the imaging instrument 100 to cause the capture of imaging information/data in the selected operational mode. In various example embodiments, step/operation 1206 may comprise at least a portion of step/operation 1304 illustrated in FIG. 13. For example, step/operation 1206 may comprise performing imaging and high-throughput screening via the imaging instrument 100. The user computing entity 10 may control the imaging instrument 100 to image the flow cell 152, and further image and/or capture image information/data while high-throughput screening is performed on the flow cell 152. For example, the imaging instrument 100 may capture information/data of molecular behavior before, during, and/or after various types of screening tests and/or under various environmental conditions (e.g., temperature, pH, and/or the like).

Returning to process 1200 of FIG. 12A, step/operation 1208 may be performed following step/operation 1206. Step/operation 1208 may comprise comparing sequencing, binding, and/or imaging data and generating a database of binding properties of molecules in the molecule library. Generating the database of binding properties may be based on comparing sequencing data, binding data, and/or imaging data. The sequencing data, binding data, and/or imaging data may be generated and/or obtained by imaging instrument 100. In various example embodiments, the sequencing data and binding data may be generated based on imaging information/data captured by the imaging instrument 100.

FIG. 12B illustrates an example of binding imaging data 1262. The binding imaging data 1262 illustrates the captured fluorescence of target molecules bound to molecules of the molecule library and/or focused library, over the at least a portion of the flow cell 152. The zoomed in binding imaging data 1262′ is a portion of the binding imaging data 1262 that is cropped and zoomed in to show individual clusters of fluorescing target molecules bound to molecules of the molecule library and/or focused library. FIG. 12B also provides an example cluster imaging representation 1264 and an example screening imaging representation 1266, according to various embodiments. The stars represent respective fluorophores. In 1264, the fluorophore is attached to the reverse complementary strand to a known 30-nt sequence (Seq 3) and in 1266 the fluorophore is coupled to a target molecule to label the target molecule.

FIG. 12C illustrates imaging data 1272, 1274, 1276, 1278 that illustrates an example of capturing imaging data related to equilibrium properties of molecules of the molecule library and/or focused library. For example, imaging data 1272 shows the results of incubating the flow cell with target molecules including an ion concentration of 0 mM of a particular target molecule. Imaging data 1272 shows a count of 0 bindings for the particular target molecule. Imaging data 1274 shows the results of incubating the flow cell with target molecules including an ion concentration of 1 mM of the particular target molecule, resulting in a determined count of approximately 8 bindings of the particular target molecule. Imaging data 1276 shows the results of incubating the flow cell with target molecules including an ion concentration of 2.5 mM of the particular target molecule, resulting in a determined count of approximately 22 bindings of the particular target molecule. Imaging data 1278 shows the results of incubating the flow cell with target molecules including an ion concentration of 5 mM of the particular target molecule, resulting in a determined count of approximately 46 bindings of the particular target molecule. Thus, equilibrium binding properties may be determined therefrom.

FIG. 12D illustrates imaging data 1282, 1284, 1286, 1288 that illustrates an example of capturing imaging data related to non-equilibrium properties of the molecule library and/or focused library. For example, at time to target molecules are provided to the flow cell 152 such that the molecule library and/or focused library and the flow cell 152 are incubated with target molecules. After a period of time, at time t₁, imaging data 1282 is captured that illustrates the equilibrium binding of the target molecules to the molecule library and/or focused library. The supply of target molecules are then turned off such that the molecular library and/or focused library and the flow cell 152 are no longer incubated in the target molecule. Imaging data 1284, 1286, and 1288 are captured at times t₂, t₃, and t₄, respectively, where to <t₁<t₂<t₃<t₄. Thus, the time-ordered sequence of imaging data 1282, 1284, 1286, 1288 illustrates the dissociation of the target molecule from molecules of the molecule library and/or focused library. Such imaging data enables, for example, the dissociation rate and/or other non-equilibrium properties to be determined.

FIG. 12E provides a plot 1280 that illustrates an example of capturing imaging data (such as the sequence of imaging data 1282, 1284, 1286, 1288) related to non-equilibrium properties of the molecule library and/or focused library at a single imaging tile and/or a single position. The plot 1280 shows the intensity of each cluster in the single imaging tile as a function of time. Plot 1281 of FIG. 12E illustrates an example of capturing imaging data (such as the sequence of imaging data 1282, 1284, 1286, 1288) related to non-equilibrium properties of the molecule library and/or focused library across multiple imaging tiles. The plot 1281 shows the intensity of each cluster in the multiple imaging tiles as a function of time.

FIG. 12F illustrates an example of step/operation 1208. The sequencing data for the molecule library and/or focused library, provided, for example, as FASTQ files, is used to generate a synthetic image based on the location information for each sequence of each cluster. FASTQ files are files that provide information in FASTQ format, which is a text-based format for storing both a biological sequence (e.g., a nucleotide sequence and/or the like) and its corresponding quality scores. The clusters in the synthetic image are shown as red crosses. The synthetic image is compared and registered with the binding and/or imaging data. The clusters in the binding and/or imaging data are shown as white dots. The sequencing data and the imaging data can be regarded to be registered if all clusters/dots in imaging data have a matching cluster/cross in the sequencing data. For example, in various embodiments, registering the imaging data enables the sequencing and binding data to be extracted from the imaging data by matching intensity measurements of the imaging data with the location of molecules of the molecule library and/or focused library such that the intensity measurements may be associated with particular molecules of the molecule library and/or focused library.

FIG. 12G provides another illustration of an example result of step/operation 1208. Each cluster in the sequence data is matched with a cluster in the binding and/or the imaging data. The intensity for each cluster in the imaging data can be extracted. The intensity can then be linked with the sequence information in the sequencing data. The distribution of intensity can then be plotted. In this example, two DNA targets are investigated at the same time. Seq 3 is 30 bp in length and the other library is pure random DNA library. The intensity distribution for these two targets is plotted. FIG. 12H illustrates an example result of the process 1200 with three DNAs of difference sequence and length. The three DNAs can be regarded as chemically distinct targets. Seq3 is 30 bp in length, Seq2 is 25 bp in length and seq1 is 20 bp in length.

In various example embodiments, the user computing entity 10 comprises means, such as processing element 12, memory 14, communications interface 16, user interface 18, and/or the like, for comparing sequencing and binding data and generating the database of binding properties. For example, the user computing entity 10 comprises a processing element 12 (e.g., a processor) for analyzing and processing the sequencing, binding, and/or imaging data and generating the database, and a memory 14 for storing the database. In various example embodiments, the database of binding properties may be generated and stored in a memory 14 of a user computing entity 10. In various other embodiments, the database of binding properties may be stored in a memory 14 of another device connected to a network 50, and the user computing entity 10 may have read and write access to the database of binding properties.

Step/operation 1208 may comprise step/operation 1306 of FIG. 13, which comprises a data analytics pipeline. The data analytics pipeline may be configured to analyze, interpret, process, and/or the like, the imaging information/data captured while observing the flow cell 152 using the imaging instrument 100. For example, the data analytics pipeline may comprise image analysis and/or image processing. In various example embodiments, image analysis and/or image processing may involve executing one or more sets of data analytics pipeline computer executable instructions to cause filtering, transforming, enhancing, and/or the like of imaging information/data captured by the imaging instrument 100. The data analytics pipeline may further comprise converting imaging information/data to a standard format. For example, imaging information/data may be converted to the FASTA format for bioinformatics processing.

The data analytics pipelines may also comprise sequence analysis. For example, imaging information/data may comprise (equilibrium and/or non-equilibrium) physical behavior or characteristics of molecules attached and/or displayed on the flow cell. The user computing entity 10 may further obtain, store, and/or otherwise have access to sequencing data for molecules attached and/or displayed on the flow cell. As such, molecular phenotypes can be correlated with molecular genotypes, and the generated database of step/operation 1206 may comprising binding properties linked to corresponding genotypes.

### 1. Exemplary Operation for Oligonucleotide Screening

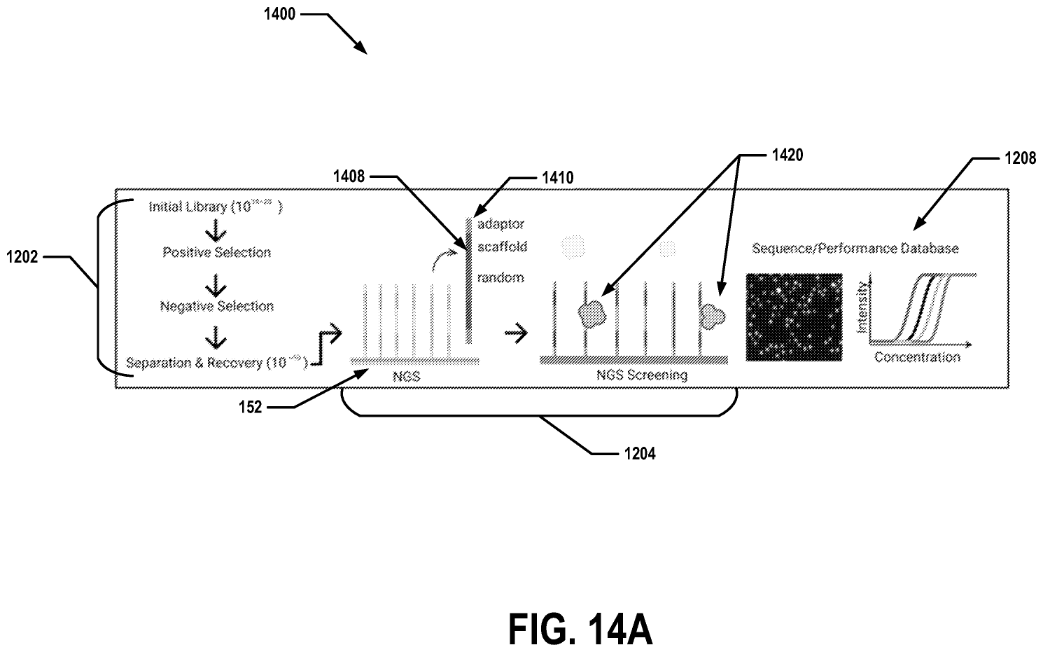

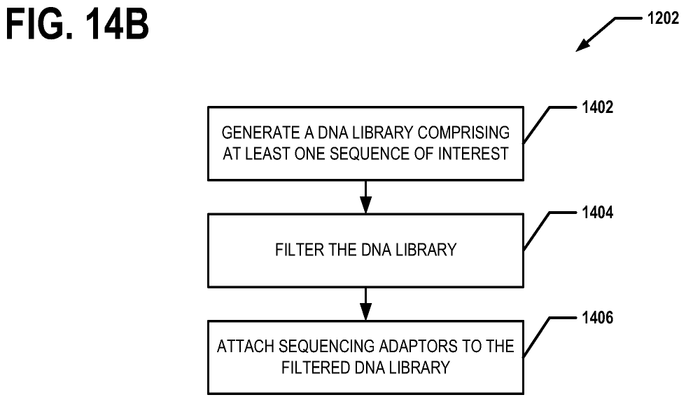

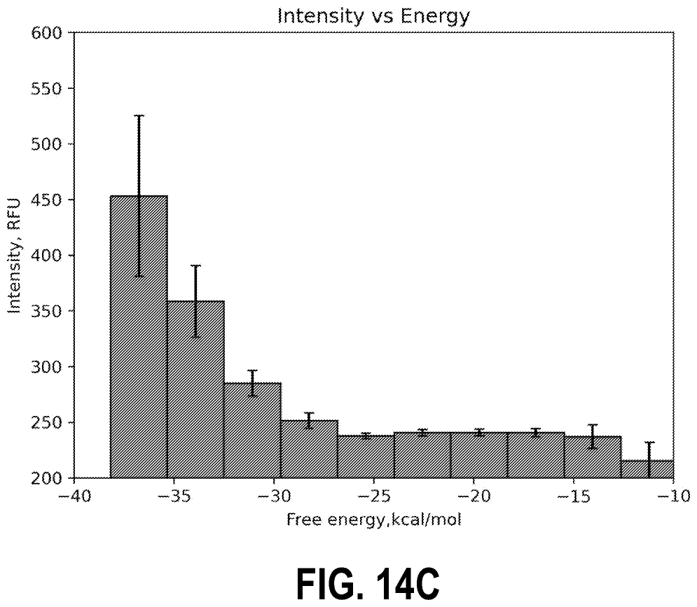

Referring now to FIG. 14A, a diagram 1400 illustrating an example process of operating the imaging instrument 100 is provided. For example, the illustrated process may be performed to at least identify, screen, and/or produce oligonucleotides (e.g., DNA aptamers), obtain imaging information/data for oligonucleotides, and/or generate a database of binding properties for oligonucleotides. In various example embodiments, oligonucleotides, such as DNA aptamers, may be capable of selectively binding with a target molecule (e.g., an ion, small molecules, nucleotides, proteins, viruses, bacteria).

The illustrated process of diagram 1400 may be an example embodiment of process 1200. Diagram 1400 illustrates at least a portion of example process 1200. For example, diagram 1400 illustrates one or more elements associated with step/operation 1202 of generating a molecule library, one or more elements associated with step/operation 1204 of attaching and sequencing the molecule library on a flow cell 152, one or more elements associated with step/operation 1206 of incubating the flow cell 152 with target molecules and observing the flow cell 152 using an imaging instrument 100, and one or more elements associated with step/operation 1208 of comparing sequencing and binding data and generating a database of binding properties of molecules of the molecule library.

Portions of example process 1200 and diagram 1400 may be specifically embodied by the example process illustrated in FIG. 14B. In various example embodiments, the example process illustrated in FIG. 14B is an embodiment of step/operation 1202 of process 1200, which generally comprises generating a molecule library. The example process illustrated in FIG. 14B begins at step/operation 1402, which comprises generating a DNA library comprising at least one sequence of interest. The generated DNA library may be referred to as the initial library and have a size of 10¹⁴⁻²³ DNA molecules, as illustrated in diagram 1400. In various example embodiments, the user computing entity 10 comprises means, such as processing element 12, memory 14, communications interface 16, user interface 18, and/or the like, for generating a DNA library comprising at least one sequence of interest. For example, the user computing entity 10 comprises a communications interface 16 for communicating with a controller of a device configured to generate a DNA library comprising at least one sequence of interest. Various steps for generating the molecule library may be performed by, for example, a human lab technician, in various embodiments.

A sequence of interest may be a DNA sequence of nucleotides (e.g., adenine, guanine, cytosine, thymine). The sequence of interest may comprise a gene relating to and/or encoding a specific physical trait (or phenotype). The sequence of interest may comprise more than one gene. In various example embodiments, the sequence of interest may be present in a subset of DNA molecules of the DNA library. In another example embodiment, the sequence of interest may be present in every DNA molecule of the DNA library. In an example embodiment, the sequence of interest may have a length up to 300 base pairs. In various example embodiments, the DNA library may be composed of single-stranded DNA (ssDNA) molecules.

Following step/operation 1402, step/operation 1404 may be performed. Step/operation 1404 comprises filtering the DNA library. In various example embodiments, the user computing entity 10 comprises means, such as processing element 12, memory 14, communications interface 16, user interface 18, and/or the like, for filtering the DNA library. For example, the user computing entity 10 comprises a communications interface 16 for communicating with a controller of a device configured to filter the DNA library. Filtering the DNA library may be performed using traditional selection methods such as particle display and/or Sequential Evolution of Ligands by Exponential Enrichment (SELEX). For example, the DNA library may be filtered using particle display by discarding a number of DNA molecules that may not, in general, satisfy some physical characteristics, such as binding with some filter molecule. For example, the DNA library may be filtered using SELEX by discarding a number of DNA molecules that do not specifically bind to a filter ligand or ligands. In various embodiments, the filtering of the DNA library may comprise positive selection and/or negative selection steps. Due to at least the filtering of the DNA library, the DNA library may have a size of around 10¹⁰ DNA molecules, such as illustrated in diagram 1400. In various example embodiments, the filtered DNA library may still comprise a subset of DNA molecules including the sequence of interest.

The example process illustrated in FIG. 14B may proceed to step/operation 1406, which comprises attaching sequencing adaptors 1410 to the filtered DNA library. In various example embodiments, the user computing entity 10 comprises means, such as processing element 12, memory 14, communications interface 16, user interface 18, and/or the like, for attaching sequencing adaptors 1410 to the filtered DNA library. For example, the user computing entity 10 comprises a communications interface 16 for communicating with a controller of a device configured to attach sequencing adaptors 1410 to the filtered DNA library, such as by introducing sequencing adaptors 1410 to the flow cell 152. Various steps for attaching the sequencing adaptors 1410 to the filtered DNA library may be performed by, for example, a human lab technician and/or a device configured to attach sequencing adaptors, in various embodiments. The sequencing adaptors 1410 may be a chain of nucleotides configured to enable sequencing, identification, and/or indexing of DNA molecules. For example, the sequencing adaptors 1410 may be configured to enable the generation of sequencing data for attached DNA molecules. Attaching sequencing adaptors 1410 to the filtered DNA library may comprise attaching the sequencing adaptors 1410 to scaffolds 1408, as illustrated in diagram 1400. In various example embodiments, scaffolds 1408 are portions of the attached DNA molecules and may comprise at least a portion of the sequence of interest.

As aforementioned, the example process illustrated in FIG. 14B may be an example embodiment of step/operation 1202, which generally comprises generating a molecule library. As a result of the example process illustrated in FIG. 14B, a filtered DNA library comprising at least one sequence of interest and having sequencing adaptors attached may be generated. Following that, the filtered DNA library comprising at least one sequence of interest and having sequencing adaptors may be attached and sequenced on a flow cell 152, such as in step/operation 1204.

In various example embodiments, step/operation 1204 of attaching and sequencing the DNA library on the flow cell 152 comprises generating sequencing data for the attached DNA library. In various example embodiments, sequencing data may be generated subsequent to the attaching of the molecule library to a flow cell 152. In various example embodiments, the user computing entity 10 comprises means, such as processing element 12, memory 14, communications interface 16, user interface 18, and/or the like, for generating sequencing data for the attached DNA library. For example, the user computing entity 10 comprises a communications interface 16 for communicating with a controller of a device configured to generate sequencing data for the attached DNA library. For example, the user computing entity 10 comprises a communications interface 16 for communicating with controller 110 of the imaging instrument 100 to generate sequencing data for the attached DNA library. For example, sequencing data for the attached DNA library may be generated by introducing fluorescently-tagged nucleotides which may competitively bind to complementary strands of attached DNA molecules, following which sequencing data comprising genetic sequences of nucleotides may be generated for each attached DNA molecule. It will be understood that generating sequencing data for the attached DNA library may also be interpreted as generating genotype data for the attached DNA molecules. For example, sequencing data may comprise genotype data, or nucleotide sequences, for attached DNA molecules.

Attaching and sequencing the DNA library on the flow cell 152 may then be followed by incubating the DNA library with target molecules 1420, as described by step/operation 1206. In the illustrated embodiment of FIG. 14A and various embodiments for identifying, screening, and/or producing oligonucleotides, the target molecules 1420 are fluorescent-labelled. In various example embodiments, the user computing entity 10 comprises means, such as processing element 12, memory 14, communications interface 16, user interface 18, and/or the like, for incubating the flow cell 152 with target molecules 1420. For example, the user computing entity 10 comprises a communications interface 16 for communicating with a controller of a device configured to incubate the flow cell 152 with target molecules 1420, such as by introducing target molecules 1420 to the flow cell 152. The target molecules 1420 may be configured to bind to attached DNA molecules, as illustrated in diagram 1400. For example, the target molecules 1420 may be nucleotides that may bind to attached DNA molecules via complementary base pairing. As another example, the attached DNA molecules may be configured to fold in a specific 3-D conformation or structure, enabling binding to target molecules 1420 such as proteins, viruses, and/or bacteria. In various example embodiments, incubating the flow cell 152 with target molecules 1420 may comprise selecting a plurality of target molecules 1420 based at least in part on the sequence of interest and/or a oligonucleotide with desired binding properties and capabilities. In various example embodiments, the target molecules 1420 bind to the attached DNA molecules at an incubated temperature, controlled by the user computing entity 10.

The target molecules 1420 may be fluorescent-labelled, and may be ions, small molecules, nucleotides, proteins, viruses, bacteria, and/or the like. Due to the fluorescent-labelling of the target molecules 1420, attached DNA molecules that bind with the target molecules 1420 may be identified by observing (e.g., using the imaging instrument 100) a change in fluorescence. As such, the molecule library is screened for molecules, or specifically in this example DNA molecules and/or aptamers, that bind to fluorescent-labelled target molecules 1420. For example, the molecule library may be screened for DNAzymes that cleave a specific nucleotide, a specific peptide, a specific protein, and/or the like by introducing the specific nucleotides, peptides, and/or proteins with fluorescent-labelling.

Following incubating the flow cell 152 and the molecule library with target molecules 1420, the flow cell 152 with attached DNA molecules and target molecules 1420 selectively bound may be observed by the imaging instrument 100, as described in step/operation 1206. Specifically, the flow cell 152, and by extension the DNA library, may be screened based on fluorescence of the fluorescent-labelled target molecules 1420. Thus, DNA molecules of the DNA library that bind with the target molecules 1420 may be identified by observing a change in fluorescence. Further, the flow cell 152, and by extension the DNA library, may be quantitatively screened based on the fluorescent intensity of the fluorescent-labelled target molecules 1420. For example, DNA molecules having a fluorescent intensity higher than a threshold intensity may be selected for further screening, processing, analysis and/or the like. Screening the DNA library may further comprise modifying the environmental conditions of the flow cell and observing fluorescent intensity changes after the modification. In an example embodiment, the flow cell 152 is observed by the imaging instrument 100 using the FP operational mode in order to image the entire flow cell 152 simultaneously. For example, using the FP operational mode of the imaging instrument 100 may enable imaging information/data to be captured at a higher speed, or have a higher temporal resolution, while still capturing data on a large portion of the flow cell 152. For example, the flow cell 152 may be imaged on a sub-minute time scale (e.g., less than a minute, less than thirty seconds, less than ten seconds, less than five seconds, approximately one second or less, and/or the like) using, for example, the FP and/or light sheet operational mode to capture imaging information indicative of non-equilibrium properties of molecules of the DNA library.

Step/operation 1208 may be performed to compare sequencing and binding data and to generate a database of binding properties for DNA molecules in the DNA library based at least in part on the observing of the flow cell 152 using the imaging instrument 100 and quantitative fluorescence screening. For example, the imaging information/data captured by the imaging instrument 100 may be analyzed, processed, and/or the like (e.g., via the data analytics pipeline) to determine binding properties and/or other characteristics for DNA molecules in the DNA library. Binding data may be generated based on captured imaging information/data. These determined binding properties and/or other characteristics may be stored in the database of binding properties. The database of binding properties for DNA molecules in the DNA library may include data on equilibrium properties (e.g., binding constants) and non-equilibrium properties (e.g., association and disassociation rates). Furthermore, such physical properties, or phenotypes, can be compared and/or linked to corresponding genetic sequences, or genotypes, due to the generation of sequencing data for attached DNA molecules. In various example embodiments, the database of binding properties may be generated using a data analytics pipeline, such as step/operation 1206 in FIG. 12A.

The quantitative fluorescence screening and the example processes performed in FIGS. 14A-B may be directed to the identification, selection, and/or production of oligonucleotides and/or allosteric nanostructures. In an example embodiment, the example processes performed in FIGS. 14A-B may be directed to the identification, selection, and/or production of DNA aptamers. For example, the example process performed in FIGS. 14A-B screen for oligonucleotides and/or DNA aptamers that bind to a target molecule 1420. Furthermore, it may be desired for the oligonucleotide or DNA aptamer to have certain binding properties and/or characteristics. As previously described, the processes illustrated in FIGS. 14A-B may be performed under different environmental conditions in order to screen for oligonucleotides and/or DNA aptamers that bind to a target molecule 1420 in specific conditions (e.g., temperature, pH).

FIG. 14C is an example of oligonucleotide screening. A known sequence of DNA is bound to a molecule library and/or focused library containing the complementary strands of the known sequence of DNA with/without mismatch. The intensity of observed fluorescence is plotted against the number of free energy. The result shows that the fluorescence intensity decreases as free energy decrease.

### 2. Exemplary Operation for Riboswitch Screening

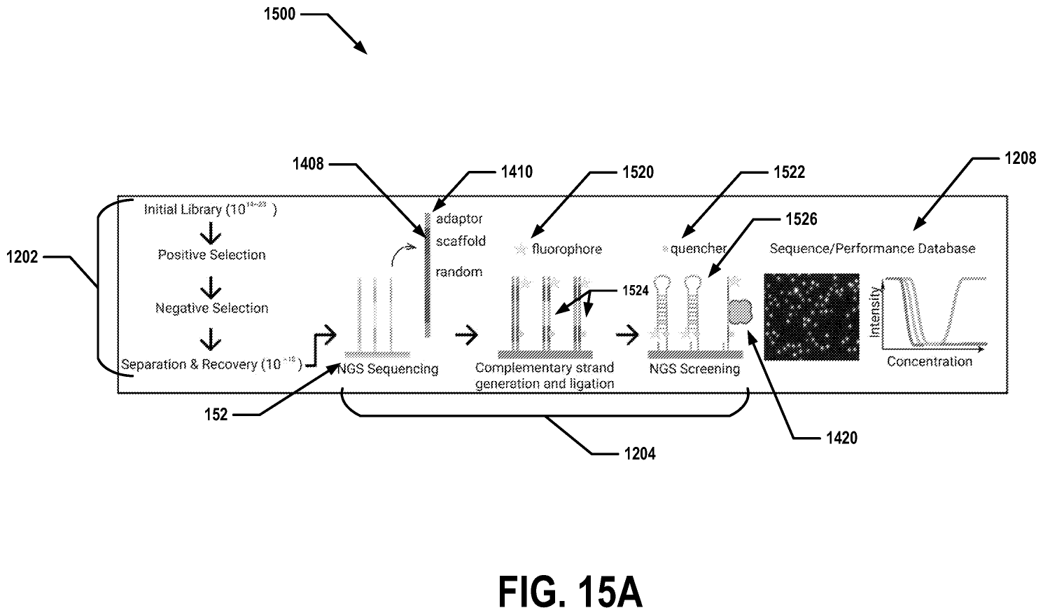

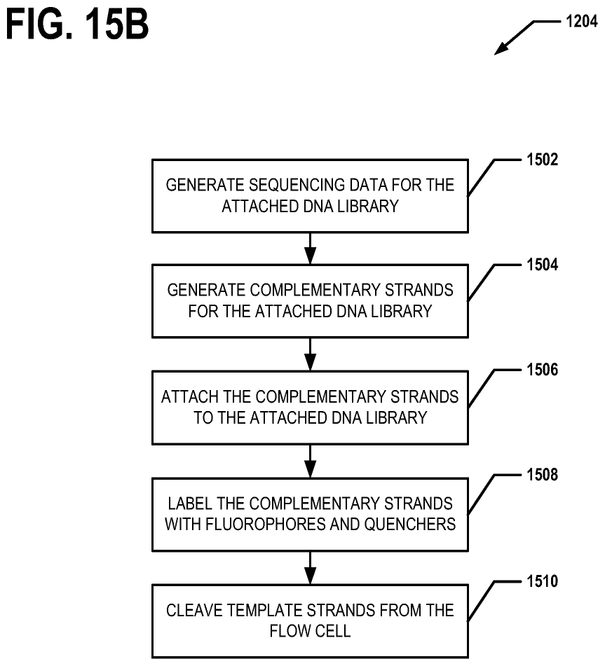

Referring now to FIG. 15A, a diagram 1500 illustrating an example process of operating the imaging instrument 100 is provided. For example, the illustrated process may be performed to at least identify, screen, and produce riboswitches and/or catalytic enzymes (e.g., DNAzyme/RNAzyme), obtain imaging information/data for riboswitches and/or catalytic enzymes, and/or generate a database of binding properties for riboswitches and/or catalytic enzyme. In various example embodiments, the catalytic enzymes may be oligonucleotides that are capable of performing and/or catalyzing a specific chemical reaction subsequent to binding with a target ligand. In various example embodiments, the catalytic enzymes are configured to “switch” between one or more conformations or structures.

The illustrated process of diagram 1500 may be an example embodiment of process 1200. Diagram 1500 illustrates at least a portion of example process 1200. For example, diagram 1500 illustrates one or more elements associated with step/operation 1202 of generating a molecule library, one or more elements associated with step/operation 1204 of attaching and sequencing the molecule library on a flow cell 152, one or more elements associated with step/operation 1206 of incubating the molecule library with target molecules and observing with an imaging instrument, and one or more elements associated with step/operation 1208 of comparing sequencing and binding data and generating a database of binding properties of molecules of the molecule library attached to the flow cell 152.

Portions of example process 1200 and diagram 1500 may be specifically embodied again by the example process illustrated in FIG. 14B. To be precise, the relevant portions of diagram 1500 relating to step/operation 1202 of example process 1200 may be embodied by FIG. 14B. For example, the illustrated process of diagram 1500 for at least producing riboswitches and/or catalytic enzymes, generating a database of binding properties for riboswitches and/or catalytic enzymes, and/or obtaining imaging information/data for riboswitches and/or catalytic enzymes may comprise steps/operations 1402, 1404, and 1406.

As described above, step/operation 1402 comprises generating a DNA library comprising at least one sequence of interest. The sequence of interest may be a DNA sequence of nucleotides. As previously described, step/operation 1404 comprises filtering the DNA library. For example, filtering the DNA library may implement methods such as particle display and SELEX. As also previously described, step/operation 1406 comprises attaching sequencing adaptors 1410 to the filtered DNA library. The sequencing adaptors 1410 may be attached to scaffolds 1408 and enable the generation of sequencing data for attached DNA molecules. In various example embodiments, the user computing entity 10 comprises means for performing steps/operations 1402, 1404, and 1406, as previously described.

The example process illustrated in FIG. 15B may be an embodiment of step/operation 1204 of process 1200, which generally comprises attaching and sequencing the molecule library on a flow cell 152. The example process illustrated in FIG. 15B begins at step/operation 1502, which comprises generating sequencing data for the attached DNA library. In various example embodiments, the user computing entity 10 comprises means, such as processing element 12, memory 14, communications interface 16, user interface 18, and/or the like, for generating sequencing data for the attached DNA library. For example, the user computing entity 10 comprises a communications interface 16 for communicating with a controller of a device configured to generate sequencing data for the attached DNA library. For example, the user computing entity 10 comprises a communications interface 16 for communicating with controller 110 of the imaging instrument 100 to generate sequencing data for the attached DNA library.

Step/operation 1502 may be followed by step/operation 1504, which comprises generating complementary strands 1524 for the attached DNA library. In various example embodiments, the user computing entity 10 comprises means, such as processing element 12, memory 14, communications interface 16, user interface 18, and/or the like, for generating complementary strands 1524 for the attached DNA library. For example, the user computing entity 10 comprises a communications interface 16 for communicating with a controller of a device configured to generate complementary strands 1524 for the attached DNA library. Complementary strands 1524 may be configured to attach to DNA molecules of the attached DNA library, via complementary base pairing. As such, at least a number of complementary strands 1524 may be generated to include a nucleotide sequence complementary to the sequence of interest. In various example embodiments, the complementary strands 1524 may be generated using DNA polymerase.

The process illustrated in FIG. 15B may then proceed to step/operation 1506, which comprises attaching the complementary strands 1524 to the attached DNA library. In various example embodiments, the user computing entity 10 comprises means, such as processing element 12, memory 14, communications interface 16, user interface 18, and/or the like, for attaching the complementary strands 1524 to the attached DNA library. For example, the user computing entity 10 comprises a communications interface 16 for communicating with a controller of a device configured to attaching the complementary strands 1524 to the attached DNA library. In various example embodiments, complementary strands 1524 may be ligated to the attached DNA library. In various example embodiments, generating the complementary strands and attaching the complementary strands may be performed at substantially the same time. For example, the complementary strands may be generated while attached to molecules of the attached DNA library.

Step/operation 1506 may then be followed by step/operation 1508, which comprises labelling the complementary strands 1524. Specifically, fluorophores 1520 and quenchers 1522 are bound to the complementary strands 1524. In various example embodiments, the user computing entity 10 comprises means, such as processing element 12, memory 14, communications interface 16, user interface 18, and/or the like, for labelling the attached DNA library. For example, the user computing entity 10 comprises a communications interface 16 for communicating with a controller of a device configured to label the complementary strands. Complementary strands 1524 may bind to both fluorophores 1520 and quenchers 1522, as illustrated in diagram 1500. In various example embodiments, the fluorophores 1520 and quenchers 1522 are bound to the complementary strands 1524 using modified nucleotides and/or short oligonucleotides. As illustrated in the diagram 1600, the fluorophores 1520 and quenchers 1522 are bound to a complementary strand 1524 at different portions of the complementary strand 1524. Thus, the quenchers 1522 are not located substantially near enough to the fluorophores 1520 to effectively quench the fluorescent intensity emitted by the fluorophores 1520.

Step/operation 1510 may then be performed following step/operation 1508. Step/operation 1510 comprises cleaving template strands from the flow cell 152. In various example embodiments, the user computing entity 10 comprises means, such as processing element 12, memory 14, communications interface 16, user interface 18, and/or the like, for cleaving template strands from the flow cell 152. For example, the user computing entity 10 comprises a communications interface 16 for communicating with a controller of a device configured to cleave template strands from the flow cell 152. The template strands are the attached DNA molecules to which complementary strands 1524 are attached. Cleaving the template strands may comprise removing, detaching, and/or the like, template strands from the flow cell 152. In various example embodiments, the template strands are cleaved using a nuclease. Subsequent to cleaving the template strands, the template strands are washed away, leaving only the complementary strands 1524 attached to the flow cell 152. Due in part to the absence of the template strands, the complementary strands 1524 may transition to another conformation or structure, such as hairpin loops 1526. Each hairpin loop 1526 comprises one complementary strand 1524, thus hairpin loops 1526 are single-stranded. Due in part to the conformation of the complementary strands 1524 into hairpin loops 1526, the quenchers 1522 are configured to quench the fluorescent intensity emitted by the fluorophores 1520. As shown in the diagram 1500, the quenchers 1522 and fluorophores 1520 of each complementary strand 1524 are located substantially nearer to each other when the complementary strand 1524 is in the hairpin loop 1526 conformation.

As previously mentioned, the example process illustrated in FIG. 15B may be an embodiment of step/operation 1204 of process 1200, which generally comprises attaching and sequencing the molecule library on a flow cell 152. As such, step/operation 1510 may be followed by incubating the flow cell 152 with target molecules 1420, as described by step/operation 1206. However, unlike the previously described processes for screening and producing oligonucleotides, target molecules 1420 are not fluorescent-labelled, in various example embodiments for screening and producing riboswitches and catalytic enzymes. In various example embodiments, the user computing entity 10 comprises means, such as processing element 12, memory 14, communications interface 16, user interface 18, and/or the like, for incubating the flow cell 152 with target molecules 1420. For example, the user computing entity 10 comprises a communications interface 16 for communicating with a controller of a device configured to incubate the flow cell 152 with target molecules 1420. For example, the flow cell 152 may be incubated at a pre-determined temperature controlled by a device configured to incubate the flow cell 152, the pre-determined temperature being a temperature promoting binding of the target molecules 1420.

The target molecules 1420 may be ions, small molecules, nucleotides, proteins, viruses, bacteria, and/or the like. As previously described, the target molecules 1420 are not fluorescent-labelled. The target molecules are configured to bind to complementary strands 1524, which may be in a hairpin loop conformation or structure 1526. As target molecules 1420 bind to complementary strands 1524, the complementary strands 1524 transition from the hairpin loop conformation 1526 to another conformation, such as an original linear conformation. For example, as shown in diagram 1500, complementary strands 1524 that bind with the target molecules 1420 are not in the hairpin loop conformation 1526. When complementary strands 1524 that bind with the target molecules 1420 transition from the hairpin loop conformation 1526 to another conformation, a change in fluorescence may be observed. For example, the fluorophores 1520 may no longer be quenched by quenchers 1522 and/or the fluorophores 1520 may be released as a result of the change in conformation, which leads to a change (e.g., an increase) in fluorescence. As such, complementary strands 1524 that bind with the target molecules 1420 may be identified, and the molecule library is screened for molecules, or specifically riboswitches and/or catalytic enzymes, that bind with the target molecules 1420. Understood otherwise, conformational or structural changes of molecules of the molecule library in response to the target molecules 1420 may be observed and screened.

Thus, over a time course of processes performed and illustrated in diagram 1500, various changes in fluorescent intensity may be observed (e.g., via imaging instrument 100) in the flow cell 152. First, an increase in fluorescent intensity, and/or a high level of fluorescent intensity, may be observed as complementary strands 1524 are labelled by fluorophores 1520 and quenchers 1522, and complementary strands 1524 are in a first conformation where the fluorescent intensity emitted by the fluorophores 1520 is not quenched by the quenchers 1522. Then, a decrease in fluorescent intensity may be observed when the template strands are cleaved and the complementary strands 1524 conform to a second conformation, such as a hairpin loop 1526, where the fluorescent intensity emitted by the fluorophores 1520 is quenched by the quenchers 1522. Finally, an increase in fluorescent intensity may be observed for complementary strands 1524 to which the target molecules 1420 bind, as the complementary strands conform to yet another conformation (e.g., the first conformation) where the fluorescent intensity emitted by the fluorophores 1520 is again not quenched by the quenchers 1522. This increase in fluorescent intensity may result in a fluorescent intensity substantially similar to the first fluorescent intensity. As such, riboswitches and/or catalytic enzymes that bind with target molecules 1420 may be identified and selected based at least in part on observing a decrease in fluorescent intensity, followed by an increase in fluorescent intensity (corresponding to binding with the target molecules 1420).

The flow cell 152 may be observed by the imaging instrument 100, as described by step/operation 1206. The process of diagram 1500 may comprise quantitative fluorescence screening based on fluorescent intensity, such as the fluorescence of fluorophores 1520 during and/or after binding of target molecules 1420. In an example embodiment, the flow cell 152 is observed by the imaging instrument 100 using the FP operational mode or the light sheet operational mode in order to image the entire flow cell 152 simultaneously. For example, using the FP operational mode and/or light sheet operational mode of the imaging instrument 100 may enable imaging information/data to be captured at a higher speed, or have a higher temporal resolution, while still capturing data on a large portion of the flow cell 152. Subsequently, step/operation 1208 of comparing sequencing data and binding data and generating a database of binding properties for riboswitches and/or catalytic enzymes may be performed.

The quantitative fluorescence screening and the example processes illustrated by FIGS. 15A-B may directed to the identification, screening, and/or production of riboswitches and/or catalytic enzymes that experience conformational or structural changes when binding with a target molecule 1420. Due to at least the quantitative fluorescence screening and the generation of the database of binding properties, DNA molecules from the DNA library with the desired nucleotide sequence and desired binding performance may be selected. As previously mentioned, the processes for identifying, screening and/or producing riboswitches and/or catalytic enzymes may be performed under different environmental conditions in order to screen for riboswitches and/or catalytic enzymes that bind to a target molecule 1420 in specific conditions (e.g., temperature, pH).

### 3. Exemplary Operation for Protein Affinity Reagent Screening

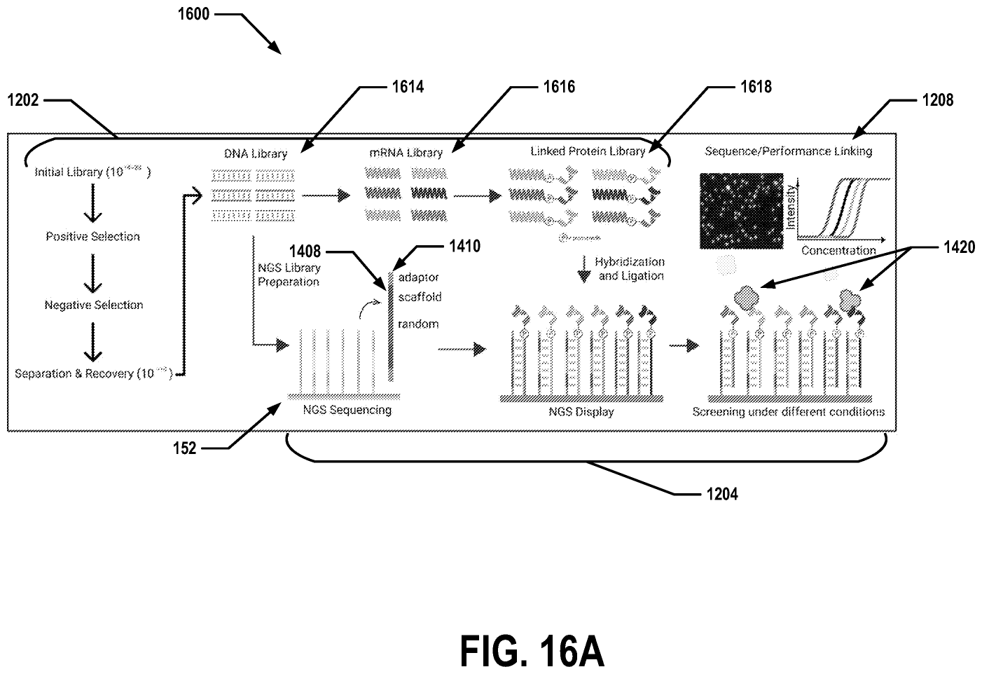

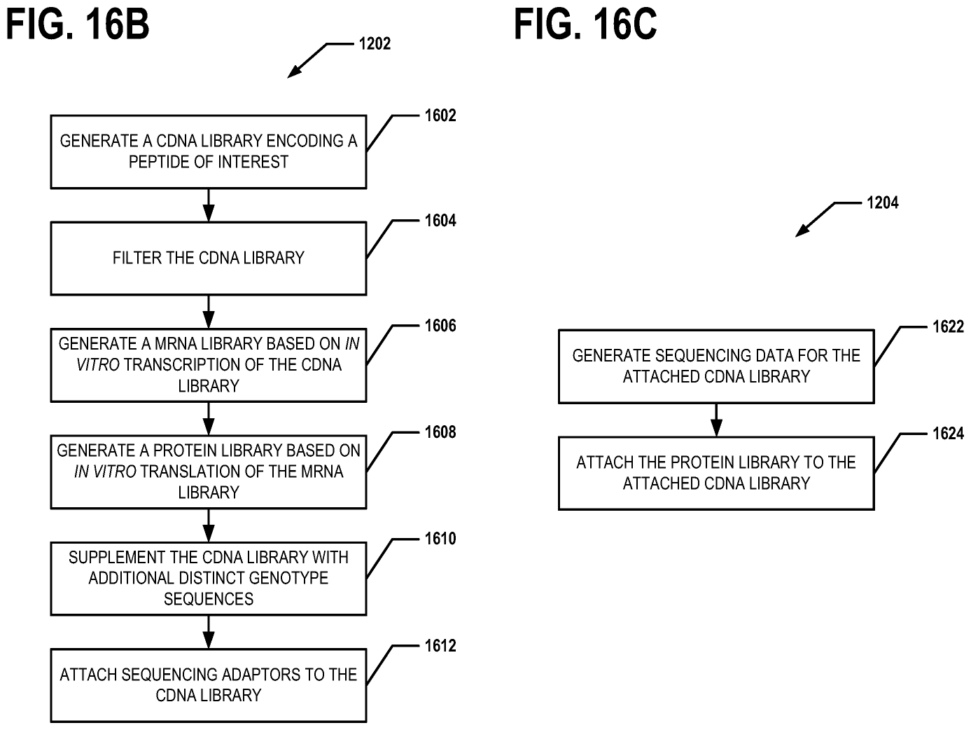

Referring now to FIG. 16A, a diagram 1600 illustrating an example process of operating the imaging instrument 100 is provided. For example, the illustrated process may be performed to at least produce affimers, such as antibodies and protein affinity reagents. The illustrated process may further obtain imaging information/data for affimers and/or generate a database of binding properties for such affimers. Affimers may be capable of binding to target molecules with a relatively high specificity and a relatively high affinity.

The illustrated process of diagram 1600 may be an example embodiment of process 1200, and diagram 1600 may illustrate at least a portion of example process 1200. For example, diagram 1600 illustrates one or more elements associated with step/operation 1202 of generating a molecule library, one or more elements associated with step/operation 1204 of attaching and sequencing the molecule library on a flow cell 152, one or more elements associated with step/operation 1206 of incubating the molecule library with target molecules and observing using an imaging instrument 100, and one or more elements associated with step/operation 1208 of generating a database of binding properties of molecules of the molecule library attached to the flow cell 152.

Portions of example process 1200 and diagram 1600 may be specifically embodied by the example process illustrated in FIG. 16B. In various example embodiments, the example process illustrated in FIG. 16B is an embodiment of step/operation 1202 of process 1200, which generally comprises generating a molecule library. The example process illustrated in FIG. 16B begins at step/operation 1602, which comprises generating a complementary DNA (cDNA) library 1614 encoding a peptide of interest. For example, the cDNA library 1614 may include a nucleotide sequence that may cause a peptide of interest to be synthesized via transcription and translation. In various example embodiments, the user computing entity 10 comprises means, such as processing element 12, memory 14, communications interface 16, user interface 18, and/or the like, for generating a cDNA library 1614 encoding a peptide of interest. For example, the user computing entity 10 comprises a communications interface 16 for communicating with a controller of a device configured to generate a cDNA library 1614 encoding a peptide of interest. Molecules of the cDNA library 1614 may comprise a 5′ untranslated region (5′ UTR). In various example embodiments, the 5′ UTR may be generated from a modified ribosome display vector (pRDV2), which may comprise a T7 promoter and a ribosome binding side (RBS). In various example embodiments, the T7 promoter may be configured to enable and/or strongly attract transcription by T7 RNA polymerase. In various example embodiments, the RBS may be configured to enable translation by ribosomes, such as E. coli ribosomes.

Step/operation 1604 may then follow step/operation 1602. Step/operation 1604 comprises filtering the cDNA library 1614. As previously described, filtering the cDNA library 1614 may implement methods such as particle display and SELEX. In various example embodiments, the user computing entity 10 comprises means, such as processing element 12, memory 14, communications interface 16, user interface 18, and/or the like, for filtering the cDNA library 1614. As noted above, in various embodiments, a human lab technician, for example, may perform various steps of filtering the cDNA library 1614. In various example embodiments, the cDNA library may be filtered to a size of around 10¹⁰ cDNA molecules.

Step/operation 1606 may then follow step/operation 1604. Step/operation 1606 comprises generating a mRNA library 1616 based on in vitro transcription of the cDNA library 1614. In various example embodiments, the user computing entity 10 comprises means, such as processing element 12, memory 14, communications interface 16, user interface 18, and/or the like, for generating a mRNA library and/or performing in vitro transcription of the cDNA library 1614. For example, in vitro transcription may comprise introducing RNA polymerase to at least a portion of the cDNA library 1614. For example, RNA polymerase may synthesize mRNA molecules based on in vitro transcription of cDNA molecules of the cDNA library 1614.

In various example embodiments, generating a mRNA library 1616 based on in vitro transcription of the cDNA library 1614 may comprise ligating puromycin to mRNA molecules of the mRNA library 1616. Puromycin may be ligated to the 3′ end of mRNA molecules. Ligating puromycin to mRNA molecules of the mRNA library 1616 may comprise first ligating DNA-puromycin linkers to the 3′ end of mRNA molecules. In various example embodiments, mRNA molecules with ligated puromycin may be referred to as mRNA molecules with pendant 3′ puromycin. It will be understood that pendant 3′ puromycin may be configured to allow mRNA molecules to covalently bond with proteins to form mRNA-protein fusions. To be exact, in vitro translation of mRNA molecules with pendant 3′ puromycin may produce a protein encoded by the mRNA molecule being attached to the same mRNA molecule via the pendant 3′ puromycin. In various example embodiments, purification may be performed subsequent to the ligating of puromycin to mRNA molecules of the mRNA library 1616. For example, purification may comprise removing or washing away molecules and/or structures used to ligate the pendant 3′ puromycin to mRNA molecules, removing or washing away unused mRNA and DNA-puromycin linkers, and/or the like

Step/operation 1608 may then follow step/operation 1606. Step/operation 1608 comprises generating a protein library based on in vitro translation of the mRNA library 1616. In various example embodiments, the user computing entity 10 comprises means, such as processing element 12, memory 14, communications interface 16, user interface 18, and/or the like, for generating a protein library and/or performing in vitro translation of the mRNA library 1616. In various example embodiments, a protein synthesis system or device may be used to perform the in vitro translation of the mRNA library 1616, such as the New England BioLabs® PURExpress® protein synthesis system. Specifically, a protein synthesis system may be used, where the system is configured to synthesize proteins in a cell-free manner from cDNA or mRNA. In an example embodiment, the protein synthesis system may comprise and/or use reconstituted and purified components used in translation in E. Coli bacteria. In various example embodiments, in vitro translation may comprise introducing transfer RNA (tRNA) molecules, ribosomes configured to translate mRNA molecules of the mRNA library 1616, and/or other molecules configured to perform and/or assist in in vitro translation.

In various embodiments, generating a protein library comprises generating a linked protein library 1618. As aforementioned and as will be understood, in vitro translation of a mRNA molecule with pendant 3′ puromycin may comprise a translating ribosome incorporating the pendant 3′ puromycin into the translated protein, thereby producing a mRNA-protein fusion molecule. It follows then that, in various example embodiment, the generated protein library may be a linked protein library 1618 containing mRNA molecules and protein molecules linked via pendant 3′ puromycin, as illustrated in diagram 1600. It may be understood that mRNA display methods or techniques may be used to generally attach a translated protein to its encoding mRNA molecule. Due to the fusion of mRNA molecules and synthesized proteins via pendant 3′ puromycin, step/operation 1608 may result in at least two molecule libraries: a cDNA library 1614 encoding a peptide of interest, and a linked protein library 1618 based on consecutive in vitro transcription and translation of the cDNA library, in an example embodiment. It will be understood that the mRNA-protein fusion library 1618 may comprise the peptide of interest being attached to its encoding mRNA molecule due to the consecutive in vitro transcription and translation.

The example embodiment of step/operation 1202 illustrated in FIG. 16B may then proceed to step/operation 1610. Step/operation 1610 comprises supplementing the cDNA library with additional distinct genotype sequences. In various example embodiments, the user computing entity 10 comprises means, such as processing element 12, memory 14, communications interface 16, user interface 18, and/or the like, for supplementing the cDNA library with additional distinct genotype sequences. For example, the user computing entity 10 comprises means for performing, and/or causing performing, of “bottleneck” PCR techniques to amplify the cDNA library 1614. In various example embodiments, performing “bottleneck” PCR techniques amplifies the cDNA library to a diversity of about 10⁶ distinct nucleotide sequences. In an example embodiment, amplification of the cDNA library is performed to a pre-determined diversity, or a pre-determined number and/or magnitude of distinct nucleotide sequences. The amplified cDNA library may comprise approximately 10 replicates of each distinct nucleotide sequence, allowing high sample sizes and redundancy when capturing information/data from the flow cell 152. For example, having 10 replicates of each distinct nucleotide sequence may enable generating an accurate binding curve measurement. In various embodiments, the entire supplemented cDNA library encodes the peptide of interest.

Following step/operation 1610, step/operation 1612 may be performed. Step/operation 1612 comprises attaching and/or ligating sequencing adaptors 1410 to the cDNA library. In various example embodiments, the user computing entity 10 comprises means, such as processing element 12, memory 14, communications interface 16, user interface 18, and/or the like, for attaching and/or ligating sequencing adaptors 1410 to the cDNA library. For example, the user computing entity 10 comprises a communications interface 16 for communicating with a controller of a device configured to attach sequencing adaptors 1410 to the cDNA library, such as at least by introducing sequencing adaptors 1410 to the flow cell 152. The sequencing adaptors 1410 may be configured to enable identification and/or indexing of cDNA molecules within the flow cell 152. For example, the sequencing adaptors 1410 may be configured to enable the generation of sequencing data for attached cDNA molecules. Attaching sequencing adaptors 1410 to the cDNA library may comprise attaching the sequencing adaptors 1410 to the scaffolds 1408, as illustrated in diagram 1600. Scaffolds 1408 may be portions of the attached cDNA molecules. In various example embodiments, scaffolds 1408 are portions of the attached cDNA molecules that encode a structural component of a protein (e.g., the peptide of interest), while the random regions of the attached cDNA molecules encode the functional component of the protein (e.g., the peptide of interest).

Referring now to FIG. 16C, an example embodiment of step/operation 1204 of process 1200 is provided. Diagram 1600 may also illustrate at least a portion of the example embodiment of step/operation 1204 illustrated in FIG. 16C. The example embodiment begins at step/operation 1622, which comprises generating sequencing data for the attached cDNA library, or cDNA molecules of cDNA library 1614 that are attached to the flow cell 152. In various example embodiments, the user computing entity 10 comprises means, such as processing element 12, memory 14, communications interface 16, user interface 18, and/or the like, for generating sequencing data for the attached cDNA library. For example, the user computing entity 10 comprises a communications interface 16 for communicating with a controller of a device configured to generate sequencing data for the attached cDNA library. For example, the user computing entity 10 comprises a communications interface 16 for communicating with controller 110 of the imaging instrument 100 to generate sequencing data from sequencing adaptors 1410 ligated to the cDNA library 1614. For example, sequencing data for the attached DNA library may be generated by introducing fluorescently-tagged nucleotides which may competitively bind to complementary strands of attached cDNA molecules, following which sequencing data comprising genetic sequences of nucleotides may be generated for each attached cDNA molecule. It will be understood that generating sequencing data for the attached cDNA library may also be interpreted as obtaining genotype data for the attached cDNA molecules. In various example embodiment, sequencing data may correlate to various sections of the flow cell 152. For example, the flow cell 152 may comprise several “lanes”, each lane comprising attached cDNA molecules including a specific nucleotide sequence.

Step/operation 1624 may then be performed. Step/operation 1624 comprises attaching, hybridizing, and/or ligating the protein library to the attached cDNA library. The protein library may be a mRNA-protein fusion library 1618. In various example embodiments, the user computing entity 10 comprises means, such as processing element 12, memory 14, communications interface 16, user interface 18, and/or the like, for attaching the mRNA-protein fusion library 1618 to the attached cDNA library. For example, the user computing entity 10 comprises a communications interface 16 for communicating with a controller of a device configured to attach the mRNA-protein fusion library 1618 to the attached cDNA library, such as by introducing the mRNA-protein fusion library to the flow cell 152. Molecules of the mRNA-protein fusion library may attach to the attached cDNA molecules on the flow cell 152 by complementary base pairing. Specifically, the mRNA component of the mRNA-protein fusion may bind to a cDNA molecule with a complementary nucleotide sequence. It may be appreciated that because each molecule of the mRNA-protein fusion library 1618 comprises a mRNA component from the mRNA library 1616, which was generated based on in vitro transcription of the cDNA library, a relatively high number of molecules of the mRNA-protein fusion library 1618 may have a complementary cDNA molecule attached to the flow cell 152 and may be able to attach to a complementary cDNA molecule.

As aforementioned, the example process illustrated in FIG. 16C may be an example embodiment of step/operation 1204 of process 1200. As such, step/operation 1624 may be followed by incubating the flow cell 152 with target molecules 1420, as described by step/operation 1206. The target molecules 1420 may be fluorescent-labelled. In various example embodiments, the user computing entity 10 comprises means, such as processing element 12, memory 14, communications interface 16, user interface 18, and/or the like, for labelling the attached DNA library. For example, the user computing entity 10 comprises a communications interface 16 for communicating with a controller of a device configured to incubate the flow cell 152 with fluorescent-labelled target molecules 1420. For example, the flow cell 152 may be incubated at a pre-determined temperature controlled by a device configured to incubate the flow cell 152. As shown in diagram 1600, fluorescent-labelled target molecules 1420 may bind to one or more protein components of mRNA-protein fusion molecules attached to attached cDNA molecules.

The target molecules 1420 may be fluorescent-labelled and may be ions, small molecules, nucleotides, proteins, viruses, bacteria, and/or the like. Due to the fluorescent-labelling of the target molecules 1420, proteins that bind with the target molecules 1420 may be identified by observing (e.g., using the imaging instrument 100) a chance in fluorescence. Furthermore, the cDNA and mRNA molecules encoding the protein may be identified due to the proteins being displayed on their encoding mRNA molecule and attached to a complementary cDNA molecule. As such, the molecule library is screened for molecules, or specifically in this example proteins and/or affimers, that bind to target molecules 1420.

The flow cell 152 may then be observed using the imaging instrument 100. Specifically, the imaging instrument may collect quantitative fluorescent intensity data from the flow cell 152 based at least in part on the fluorescence emitted by the target molecules 1420. In other words, the process of diagram 1600 may comprise quantitative fluorescence screening based on the fluorescent intensity of the target molecules 1420. In an example embodiment, the flow cell 152 is observed by the imaging instrument 100 using FP mode in order to image the entire flow cell 152 simultaneously. The previously generated sequencing data and the observed fluorescent intensity data may be compared and used to generate a protein binding property database for the cDNA library 1614 and/or the linked protein library 1618.

Example embodiments of the present disclosure may be directed to the identification, screening, and/or production of affimers such as antibodies and protein affinity reagents by using quantitative fluorescence screening and the example processes illustrated by FIGS. 16A-C. Due to at least the quantitative fluorescence screening and the generation of the database of binding properties, affimers encoded by the cDNA library with the desired properties (e.g., thermal stability, pH stability) may be selected. Furthermore, cDNA and mRNA molecules that encode for selected proteins and/or affimers and/or contain a sequence of interest encoding selecting proteins and/or affimers may be identified. As previously described, the processes illustrated in FIGS. 16A-C may be performed under different environmental conditions in order to screen for proteins and/or affimers that bind to a target molecule 1420 in specific conditions (e.g., temperature, pH) with specific thermal and pH stability.

### 4. Exemplary Operation for Enzyme Screening

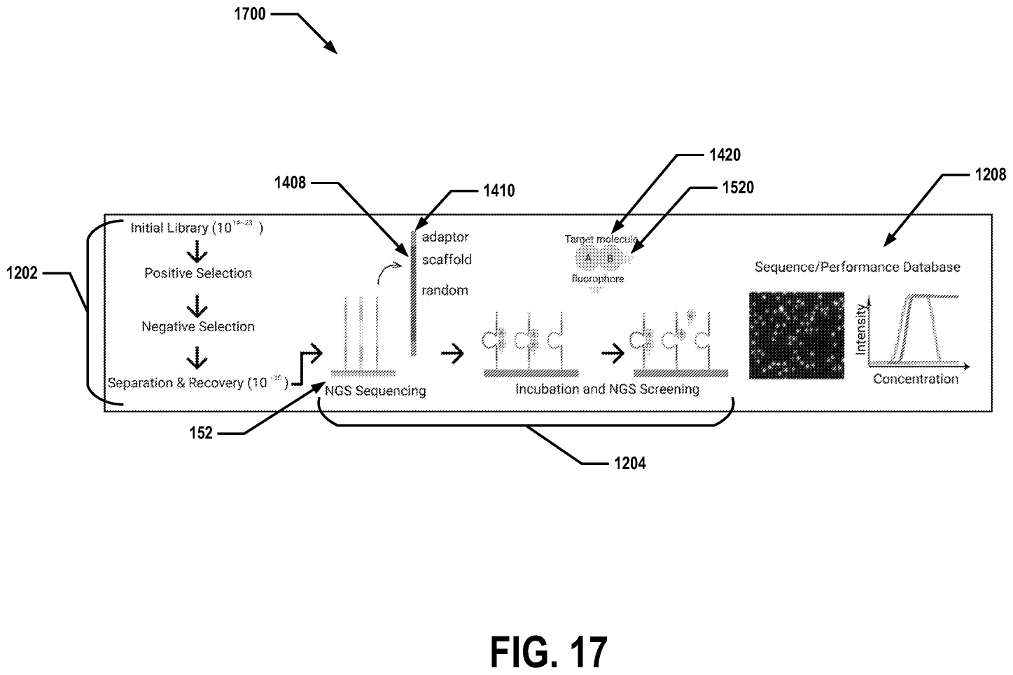

Referring now to FIG. 17, a diagram 1700 illustrating an example process of operating the imaging instrument 100 is provided. For example, the illustrated process may be performed to at least identify, screen, and/or produce enzymes, such as peptide enzymes, protein enzymes, deoxyribozymes (DNAzyme), and ribozymes. The illustrated embodiment of FIG. 17 is directed to screening for DNAzyme and/or RNAzyme.

The illustrated process of diagram 1700 may be an example embodiment of process 1200, and diagram 1700 may illustrate at least a portion of example process 1200. For example, diagram 1700 illustrates one or more elements associated with step/operation 1202 of generating a molecule library, one or more elements associated with step/operation 1204 of attaching and sequencing the molecule library on a flow cell 152, one or more element associated with step/operation 1206 of incubating the molecular library with target molecules and observing using an imaging instrument 100, and one or more elements associated with step/operation 1208 of generating a database of binding properties of molecules of the molecule library attached to the flow cell 152.

Diagram 1700 first illustrates an example process of generating a molecule library, in accordance with step/operation 1202. An initial library of a size of approximately 10¹⁴⁻²³ molecules is generated, and filtered with positive and negative selection to result in a filtered library of a size of approximately 10¹⁰ molecules. The initial library is composed of molecules from which enzymes may be identified, screened, and/or produced. For example, diagram 1700 illustrates generating an enzyme library. In the illustrated embodiment for screening DNAzyme and/or RNAzyme, the initial library, and by extension the filtered (e.g., generated) molecule library may be composed of DNA (e.g., ssDNA, cDNA) molecules and/or RNA (e.g., mRNA) molecules. Sequencing adaptors 1410 may also be attached to molecules of the filtered (e.g., generated) molecule library. As previously mentioned, sequencing adaptors 1410 may be attached to scaffolds 1408. In various example embodiments for identifying, screening, and/or producing enzymes, the scaffolds 1408 include peptide enzymes, protein enzymes, deoxyribozymes and/or ribozymes. For example, in the illustrated embodiment, the scaffolds 1408 include DNAzymes and/or ribozymes. It may be appreciated that a process of generating a molecule library for identifying, screening, and/or producing enzymes may resemble the example embodiment of step/operation 1202 illustrated in FIG. 14B, which comprises the steps/operations 1402, 1404, and 1406 of generating a DNA library, filtering the DNA library, and attaching sequencing adaptors 1410 to the DNA library, respectively. In various example embodiments, the user computing entity 10 comprises means, such as processing element 12, memory 14, communications interface 16, user interface 18, and/or the like, for generating a molecule library and/or various steps/operations for generating the molecule library.

Diagram 1700 further illustrates an example process of attaching and sequencing the molecule library on a flow cell 152, in accordance with step/operation 1204. As previously described in example embodiments of step/operation 1204, the molecule library may be attached to the flow cell 152, and subsequently, sequencing data for the attached molecule library may be generated. In the illustrated embodiment, sequencing data may be generated in a similar manner as in step/operation 1502 of FIG. 15B and step/operation 1622 of FIG. 16C; however, it will be understood that other methods for generating sequencing data for the attached molecule library may be used. As previously described, sequencing data may be generated based at least in part on the sequencing adaptors 1410.

Subsequent to generating sequencing data, target molecules 1420 may be introduced (e.g., via fluid flow) to the flow cell 152, and the flow cell 152 may be incubated. The target molecules 1420 are fluorescent-labelled, such as with fluorophores 1520. As illustrated in diagram 1700, a target molecule 1420 may be a molecule configured to be catalyzed, cleaved, ligated, and/or the like by enzymes, such as the attached molecules of the molecule library. For example, a target molecule 1420 may be composed of two or more components (e.g., components A and B), which may be cleaved by DNAzymes attached to the flow cell 152. It may be appreciated that fluorophores 1520 may be attached only to one component of a target molecule 1420 (e.g., fluorophores 1520 attached to component B of target molecule 1420). As the attached molecules on the flow cell 152 interact with the fluorescent-labelled target molecules 1420, a change in fluorescent intensity emitted by the fluorophores 1520 may be observed. Specifically, the attached enzymes may be configured to cleave the target molecules 1420 such that fluorophores 1520, or components of target molecules 1420 to which fluorophores 1520 are attached, are released and washed away. Therefore, a decrease in fluorescent intensity for attached enzymes that bind to the target molecules 1420 may be observed. In various example embodiments, the user computing entity 10 comprises means, such as processing element 12, memory 14, communications interface 16, user interface 18, and/or the like, for introducing target molecules 1420 tagged with fluorophores 1520 into the flow cell 152.

Thus, over a time course of processes performed and illustrated in diagram 1700, various changes in fluorescent intensity may be observed (e.g., via imaging instrument 100) in the flow cell 152. First, an increase in fluorescent intensity may be observed for an enzyme that binds with a fluorescent-labelled target molecule 1420. On the other hands, no change in fluorescent intensity may be observed for an enzyme that does not bind with the fluorescent-labelled target molecule 1420. Then, a decrease in fluorescent intensity may be observed for an enzyme that binds with a fluorescent-labelled target molecule 1420 and that catalyzes, cleaves, ligates, and/or otherwise interacts with the fluorescent-labelled target molecule 1420. As such, enzymes that perform a specific function on a target molecule 1420 may be identified and selected based at least in part on observing first an increase in fluorescent intensity (corresponding to binding with the target molecule 1420), followed by a decrease in fluorescent intensity (corresponding to interacting with the target molecules 1420).

The flow cell 152 may be observed using the imaging instrument 100. Specifically, the imaging instrument may collect quantitative fluorescent intensity data from the flow cell 152 based at least in part on the fluorescence emitted by fluorophores 1520 attached to the target molecules 1420. In other words, the process of diagram 1700 may comprise quantitative fluorescence screening based on changes to fluorescent intensity as enzymes of a molecule library bind with target molecules 1420. In an example embodiment, the flow cell 152 is observed by the imaging instrument 100 using FP mode in order to image the entire flow cell 152 simultaneously. The previously generated sequencing data and the observed fluorescent intensity data may be compared and used to generate an enzymatic performance database for the enzyme molecule library.

Example embodiments of the present disclosure may be directed to the identification, screening, and/or production of enzymes, such as DNAzymes and/or RNAzymes. Due to at least the quantitative fluorescence screening and the generation of the database of binding properties, enzymes with the desired properties (e.g., thermal stability, pH stability) may be selected. As previously described, the processes illustrated in FIG. 17 may be performed under different environmental conditions in order to screen for enzymes that bind to a target molecule 1420 in specific conditions (e.g., temperature, pH), or enzymes with desired thermal and pH stability.

### 5. Exemplary Operation for Biomolecule Conjugated Nanomaterial Screening

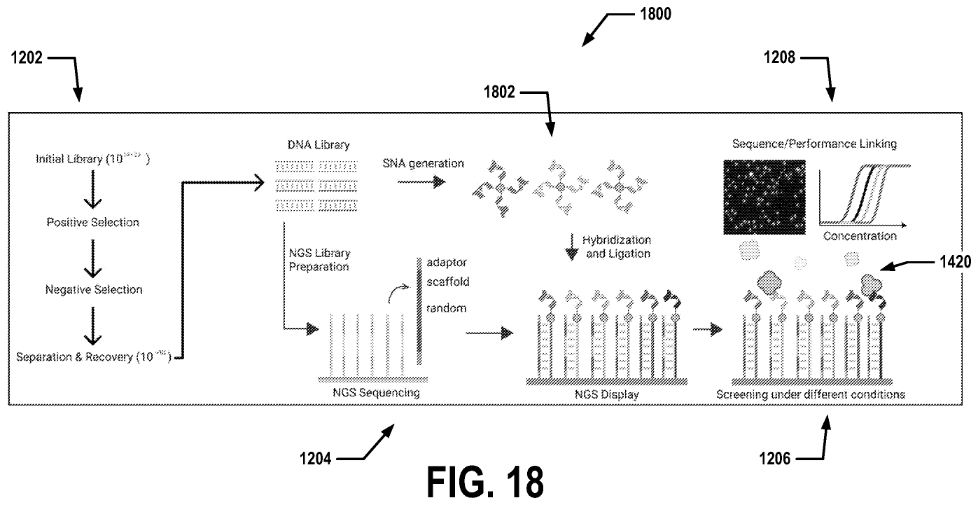

Referring now to FIG. 18, a diagram 1800 illustrating an example process of operating the imaging instrument 100 is provided. For example, the illustrated process may be performed to at least identify, screen, and/or produce biomolecule conjugated nanomaterials, such as spherical nucleic acids (SNAs), DNA-silica nanocomposites. The illustrated embodiment of FIG. 18 is directed to screening for DNA or RNA conjugated gold nanoparticles.

The illustrated process of diagram 1800 may be an example embodiment of process 1200, and diagram 1800 may illustrate at least a portion of example process 1200. For example, diagram 1800 illustrates one or more elements associated with step/operation 1202 of generating a molecule library, one or more elements associated with step/operation 1204 of attaching and sequencing the molecule library on a flow cell 152, one or more element associated with step/operation 1206 of incubating the molecular library with target molecules and observing using an imaging instrument 100, and one or more elements associated with step/operation 1208 of generating a database of binding properties of molecules of the molecule library attached to the flow cell 152.

The process starts with a initial library of size 10¹⁴-10¹⁸, and then the size of the library is reduced through preselection methods including positive selection and negative selection methods. The focused library can be separated and recovered with size of about 10¹⁰ which is suitable for sequencing. Then a portion of the focused library is sequenced and immobilized on a flow cell 152. Another portion of the library is used to generate a spherical nucleic acid (SNA) library 1802. The SNA library 1802 comprises a plurality of gold nanoparticles is that have each been conjugated with one unique DNA sequence and replication thereof. Then the generated SNA library 1802 will be hybridized to the DNA library immobilized on surface through DNA hybridization. Labeled targets can then be flowed into the flow cell and incubate with the library. The flow cell is then viewed using the optical instrument to generate the performance/imaging data. The imaging database is then linked with the sequencing database and a function landscape can be generated. For example, the observed performance/imaging data can be registered to the known locations of various clusters of the immobilized portion of the library and information regarding the targets can be extracted from the performance/imaging data based on the registration thereof.

### 6. Exemplary Operation for Imaging a Flow Cell For Biomolecule Screening

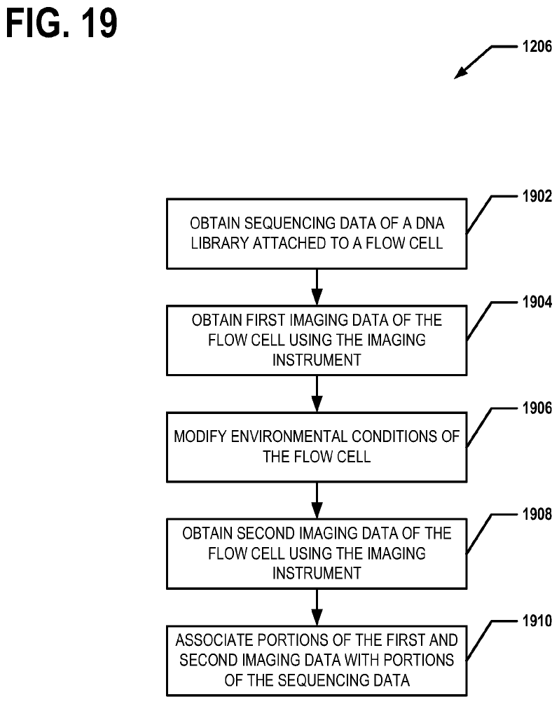

Referring now to FIG. 19, an example process of operating the imaging instrument 100 is provided. Specifically, FIG. 19 illustrates an example embodiment of step/operation 1206 of process 1200, which comprises observing the flow cell 152 using the imaging instrument 100. For example, the illustrated example embodiment of step/operation 1206 may be performed subsequent to any example embodiment of step/operation 1204, such as example embodiments illustrated in 15B and 16C. In various example embodiments, the illustrated embodiment of step/operation 1206 in FIG. 19 enables the production of various molecules such as oligonucleotides, riboswitches, catalytic enzymes, affimers, and/or the like, observing the presence, binding properties, and/or behavior of molecules displayed on the flow cell 152 under different environmental conditions of the flow cell 152. For example, the illustrated embodiment of step/operation 1206 in FIG. 19 may generally employ directed evolution philosophy to obtain information and/or produce molecules with desired properties and/or behaviors.

The illustrated example embodiment of step/operation 1206 may begin at step/operation 1902, which comprises obtaining sequencing data of a DNA library displayed on a flow cell 152. In the example embodiment illustrated in FIG. 16A, the DNA library is a cDNA library 1614. In various example embodiments, the user computing entity 10 may comprise means, such as processing element 12, memory 14, communications interface 16, and/or user interface 18, for obtaining sequencing data of a DNA library displayed on a flow cell 152. For example, the user computing entity 10 may comprise communications interface 16 for obtaining (e.g., receiving) sequencing data of a DNA library attached to a flow cell 152. For example, the user computing entity 10 may obtain (e.g., retrieve) previously generated sequencing data of a DNA library from a memory 14. For example, the user computing entity 10 may obtain (e.g., generate) sequencing data via an imaging instrument 100. As previously mentioned, sequencing data may comprise specific genotype data (e.g., nucleotide sequences) for specific DNA molecules within the DNA library.

Step/operation 1904 may follow step/operation 1902. Step/operation 1904 comprises obtaining first imaging data of the flow cell 152 using the imaging instrument 100. In various example embodiments, the user computing entity 10 comprises means, such as processing element 12, memory 14, communications interface 16, and/or user interface 18, for obtaining first imaging data of the flow cell 152 using the imaging instrument 100. For example, the user computing entity 10 may comprise communications interface 16 for controlling the imaging instrument 100 (e.g., via a controller 110) to obtain first imaging data of the flow cell 152. For example, the first imaging data may be captured while first environmental conditions (e.g., temperature pH, and/or the like) are present in the flow cell 152. For example, the user computing entity 10 may obtain (e.g., retrieve) first imaging data captured by the imaging instrument 100 from memory 14. In various embodiments, the first imaging data comprises and/or indicates information regarding the equilibrium and/or non-equilibrium properties of the displayed molecules under first environmental conditions. In various example embodiments, the first imaging data may be obtained (e.g., captured) by an imaging instrument 100 using the FP operational mode or the light sheet operational mode. For example, using the FP operational mode or light sheet operational mode of the imaging instrument 100 may enable imaging information/data to be captured at a higher speed, or have a higher temporal resolution, while still capturing data on a large portion of the flow cell 152.

Step/operation 1906 may then follow step/operation 1904. Step/operation 1906 comprises modifying environmental conditions of the flow cell 152. In various example embodiments, the user computing entity 10 comprises means, such as processing element 12, memory 14, communications interface 16, and/or user interface 18, for modifying environmental conditions of the flow cell 152. For example, the user computing entity 10 may comprise communications interface 16 for communicating with a controller of a device (e.g., an incubator) to modify environmental conditions of the flow cell 152. For example, the environmental conditions within the flow cell 152 may be evolved from the first environmental conditions to second environmental conditions. In various embodiments, at least one aspect of the first environmental conditions differs from the corresponding aspect of the second environmental conditions. In various example embodiments, environmental conditions such as temperature, pH, and/or the like may be modified. In various example embodiments, environmental conditions may be modified based on sections of the flow cell 152. For example, a first section of the flow cell 152 may have, and/or be modified to have a first temperature, and a second section of the flow cell 152 may have, and/or be modified to have a second temperature.

Following step/operation 1906, step/operation 1908 may be performed. Step/operation 1908 comprises obtaining second imaging data of the flow cell 152 using the imaging instrument 100. In various example embodiments, the user computing entity 10 comprises means, such as processing element 12, memory 14, communications interface 16, and/or user interface 18, for obtaining second imaging data of the flow cell 152 using the imaging instrument 100. For example, the user computing entity 10 may comprise communications interface 16 for controlling the imaging instrument 100 (e.g., via a controller 110) to obtain second imaging data of the flow cell 152. For example, the second imaging data may be captured while second environmental conditions (e.g., temperature pH, and/or the like) are present in the flow cell 152. For example, the user computing entity 10 may obtain (e.g., retrieve) second imaging data captured by the imaging instrument 100 from memory 14. In various embodiments, the second imaging data comprises and/or indicates information regarding the equilibrium and/or non-equilibrium properties of the displayed molecules under first environmental conditions. In various example embodiments, the second imaging data may be obtained (e.g., captured) by an imaging instrument 100 using the FP operational mode or the light sheet operational mode. For example, using the FP operational mode or light sheet operational mode of the imaging instrument 100 may enable imaging information/data to be captured at a higher speed, or have a higher temporal resolution, while still capturing data on a large portion of the flow cell 152.

It will be understood that the second imaging data may exhibit differences from the first imaging data due at least in part to the modification of environmental conditions of the flow cell 152 (see step/operation 1906). For example, in the illustrated embodiment of diagram 1600, a subset of proteins to which fluorescent-labelled target molecules 1420 are bound may have a lower molecular stability at a modified environmental condition (e.g., temperature, pH) of the flow cell 152 relative to a molecular stability at the original environmental condition. Thus, the subset of proteins and/or the fluorescent-labelled target molecules 1420 may be washed away due to the fluid flow in the flow cell 152, resulting in only proteins with higher stability at the modified environmental condition remaining displayed on the flow cell 152. In at least this manner, molecules with desired properties and characteristics may be directly selected, according to a direct evolution philosophy.

Following step/operation 1908, step/operation 1910 may be performed. Step/operation 1910 comprises associating portions of the first and second imaging data with portions of the sequencing data. In various example embodiments, the user computing entity 10 comprises means, such as processing element 12, memory 14, communications interface 16, and/or user interface 18, for associating portions of the first and second image data with portions of the sequencing data. For example, the user computing entity 10 may comprise processing element 12 for associating portions of the first and second image data with portions of the sequencing data. Portions of the first imaging data, portions of the second imaging data, and portions of the sequencing data may be associated with each other relative to the flow cell 152, or sections of the flow cell 152.

As previously mentioned, the example process illustrated in FIG. 19 may be an example embodiment of step/operation 1206 of process 1200. For example, the example process comprising steps/operations 1902, 1904, 1906, 1908, and 1910 may be directed to observing the flow cell 152 using an imaging instrument 100. Following the example process illustrated in FIG. 19 then, a database of binding properties may be generated for molecules of the molecule library. Specifically, the database of binding properties may comprise sequencing data for molecules of the molecule library. That is, the database may store binding properties for each molecule as well as sequencing data, such as nucleotide sequences that may encode or configure such binding properties. In other words, the database may maintain a phenotype-genotype linkage for each molecule in the molecule library.

### 7. Exemplary Operation for Imaging a Flow Cell for Determination of Biomolecule Properties

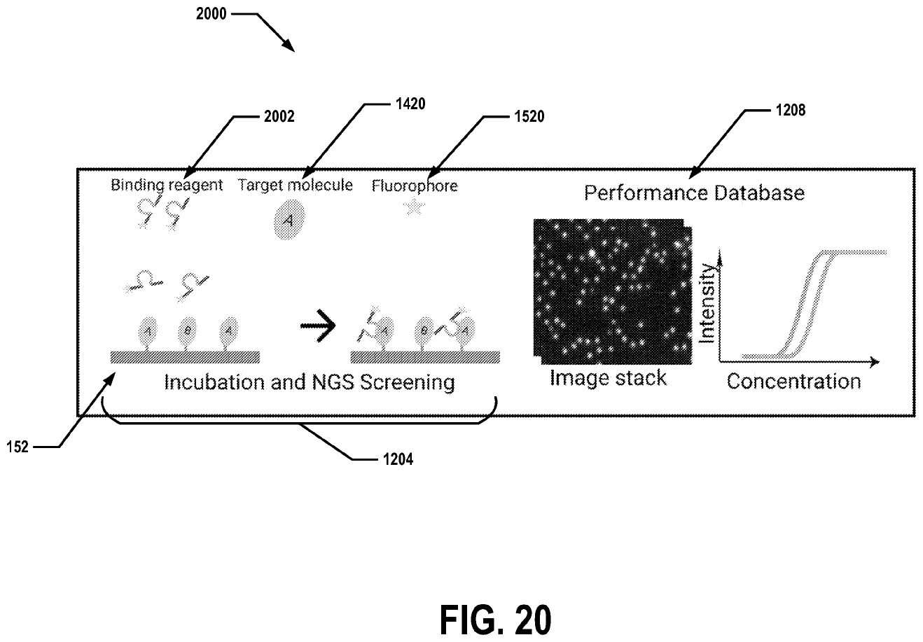

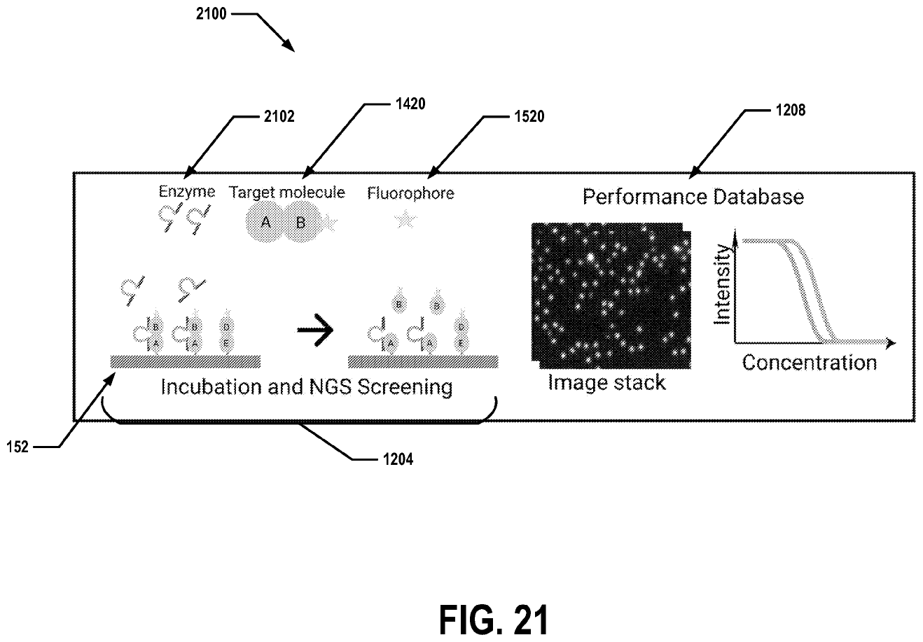

FIGS. 20 and 21 each provide diagrams illustrating example processes of operating the imaging instrument 100. Diagrams 2000 and 2100 both illustrate processes that may be used to determine properties of biomolecules under specifically designed conditions. The biomolecules may be affinity reagents or binding reagents (e.g., DNA/RNA aptamers, peptide/protein binders, affimers, antibodies, and/or the like), enzymes (e.g., deoxyribozymes, ribozymes, peptide enzymes, protein enzymes, and/or the like), riboswitches, and/or the like.

Referring first to FIG. 20, diagram 2000 illustrates an example process for determining binding properties of one or more affinity or binding reagents 2002. The example process may specifically be used to determine properties such as equilibrium properties (e.g., binding constant, and/or the like), non-equilibrium properties (e.g., association rate, dissociation rate, and/or the like), effects of environmental conditions on binding (e.g., temperature, pH, and/or the like), specificity (e.g., target specificity, site specificity, and/or the like), and/or the like.

First, target molecules 1420 may be attached, conjugated, and/or the like, to the flow cell 152. One or more types of target molecules 1420 may be attached to the flow cell 152. For example, in the illustrated embodiment, target molecules 1420 of type “A” and target molecules 1420 of type “B” are attached to the flow cell 152. It will be understood that more than two types of target molecules 1420 may be attached to the flow cell 152. The affinity reagents 2002 may be configured to only bind with one type of target molecule 1420. In the illustrated embodiment, the affinity reagents 2002 may only bind with target molecules 1420 of type “A”. Thus, a first type of target molecules 1420 that bind with the affinity reagents 2002 (e.g., type “A”) may serve as a positive control to determine the equilibrium and non-equilibrium binding properties of the affinity reagents 2002. Meanwhile, other types of target molecules 1420 that do not bind with affinity reagents 2002 (e.g., type “B”), but may have similar structure to the first type of target molecules 1420, may serve as a negative control to determine the specificity of the affinity reagents 2002.

The affinity reagents 2002 may also be tagged or labelled with fluorophores 1520. The affinity reagents 2002 are introduced to the flow cell 152, and the fluorescent intensity emitted by the fluorophores 1520 are observed (e.g., via the imaging instrument 100). An increase in fluorescent intensity may be observed for the first type of target molecules 1420 (e.g., type “A”) as affinity reagents 2002 bind with them, while no change in fluorescent intensity may be observed for the other types of target molecules 1420. In an example embodiment, the flow cell 152 is observed by the imaging instrument 100 using FP mode in order to image the entire flow cell 152 simultaneously. The observed fluorescent intensity data may be used to generate a performance database, and the properties of the affinity reagents 2002 may be determined.

Referring now to FIG. 21, diagram 2100 illustrates an example process for determining catalytic properties of one or more enzymes 2102. The example process may specifically be used to determine properties such as catalytic properties (e.g., catalytic performance efficiency, and/or the like), specificity (e.g., target specificity, site specificity, and/or the like), effects of environmental conditions on binding (e.g., temperature, pH, molecular composition of an environment/fluid within the flow cell 152, and/or the like), and/or the like.

First, target molecules 1420 may be attached, conjugated, and/or the like, to the flow cell 152. The target molecules 1420 are fluorescent-labelled, such as with fluorophores 1520. One or more types of target molecules 1420 may be attached to the flow cell 152, and each target molecule 1420 may comprise two or more components that may be catalyzed, cleaved, ligated, and/or the like, by enzymes 2102. For example, a first type of target molecule 1420 may comprise an “A” and a “B” component, while another type of target molecule 1420 may comprise a “D” and an “E” component. Each target molecule 1420 may have fluorophores 1520 attached to only one component. It will be understood that more than two types of target molecules 1420 may be attached to the flow cell 152. The enzymes 2102 may be configured to only bind with one type of target molecule 1420. In the illustrated embodiment, the enzymes 2102 bind with a first type of target molecules 1420 that comprise an “A” and a “B” component. Thus, the first type of target molecules 1420 serve as a positive control to determine catalytic properties of the enzymes 2102. Meanwhile, the enzymes 2102 do not bind with other types of target molecules 1420, such as target molecules 1420 that comprise a “D” and an “E” component. As such, other types of target molecules 1420 that do not bind with enzymes 2102, but may have similar structure with the first type of target molecules 1420, may serve as a negative control to determine the specificity of the enzymes 2102.

The enzymes 2102 are introduced to the flow cell 152, and the flow cell 152 is observed. In various example embodiments, the flow cell 152 is observed by the imaging instrument 100 using FP mode in order to image the entire flow cell 152 simultaneously. As the enzymes 2102 flow into the flow cell 152, the enzymes 2102 bind to the first type of target molecules 1420 and interact (e.g., catalyze, cleave, ligate, and/or the like) with the first type of target molecules 1420 such that a component of the target molecules 1420 to which fluorophores 1520 are attached is released and washed away. As such, a decrease in fluorescent intensity for target molecules 1420 to which the enzymes 2102 bind may be observed, while no change in fluorescent intensity for target molecules 1420 to which the enzymes 2102 do not bind may be observed. The observed fluorescent intensity data may be used to generate a performance database, and the properties of the enzymes 2102 may be determined.

### I. Technical Advantages

Various embodiments of the present invention provide technical advantages. As previously described, various embodiments provide an imaging instrument 100 configured to capture imaging information/data using various operational modes. For example, the TIRF operational mode provides improved spatial resolution of features on a surface, such as a flow cell 152. Additionally, the Fourier ptychography (FP) operational mode and/or light sheet operational mode enables the imaging instrument 100 to capture imaging data having a sub-minute sampling rate (e.g., a sampling rate of less than a minute, less than thirty seconds, less than ten seconds, less than five seconds, approximately one second or less, and/or the like) which allows the imaging instrument 100 to capture imaging data that encodes non-equilibrium properties of the displayed molecules. Using the imaging instrument 100 in the FP operational mode and/or light sheet operational mode may produce wide-filed, high resolution complex sample images with, for example, a sub-minute sampling rate. Due to the ability of the imaging instrument 100 in FP operational mode and/or light sheet operational mode to capture band-limited sample images in Fourier space and produce wide-filed, high resolution complex sample images, a large area of a flow cell 152 may be imaged. Using the imaging instrument 100 in FP operational mode or light sheet operational mode may be particularly advantageous when capturing data on non-equilibrium molecular functions of molecules in a flow cell 152. In particular, non-equilibrium molecular functions may occur on very short time scales (e.g., less than a minute, less than thirty seconds, less than ten seconds, less than five seconds, approximately one second or less, and/or the like). As such, the imaging instrument 100 in FP operational mode and/or light sheet operational mode is uniquely capable of capture imaging information/data comprising non-equilibrium molecular functions.

In various embodiments, the imaging instrument 100 is configured to be operated in a time delay imaging operational mode. The time delay imaging operational mode enables fast scanning of the entire sequencing flow cell which allows the imaging instrument 100 to capture imaging data that encodes non-equilibrium properties of the displayed molecules. The high scanning speed or the high sample movement speed is achieved by effective integration time. The time delay imaging operational mode can also be used with TIRF imaging to improve the spatial resolution. Thus, the time delay imaging operational mode provides technical improvements over conventional imaging techniques and enables exploration and determination of non-equilibrium properties of the displayed molecules.

Furthermore, various embodiments of the present disclosure provide a database of binding properties of molecules in a molecule library. Specifically, the database of binding properties may further comprise sequencing data associated with the binding properties. By providing associated sequencing data, or genotype, for various binding properties, molecules with the desired binding properties can be synthesized more accurately. For example, such a database with genotype-phenotype linkage may be used directly for reagent development or as a starting library to be used for further rounds of selection and screening. As the physical genotype-phenotype linkage is maintained throughout the imaging process, molecular properties may be more efficiently and more completely determined.

Various embodiments of the present disclosure additionally and advantageously provide methods for functional selection of molecules from a molecule library using positive and/or negative selection steps. Due to the molecules attached to and/or displayed on the flow cell 152 being characterized by observing various binding characteristics, molecule selection may be functional. For example molecules may be selected based on behavior with allosteric switching and/or enzymatic activity. Furthermore, molecule selection/screening may be additionally and/or alternatively based on non-equilibrium parameters. For example, an initial library may be filtered, such as in step/operation 1404 or step/operation 1604 in embodiments of generating a molecule library. Such filtering, also referred to as a down selection herein, may be based on non-equilibrium parameters and characteristics.

Various embodiments of the present disclosure further provide methods directed to characterizing a molecule library in a multiplexed or parallel manner. For example, the molecule library may be a library of random peptides, and by introducing labelled and/or fluorescent-labelled antibodies, the types of antibodies present in the sample may be inferred. As such, various example embodiments of the present disclosure may be used as a multiplexed serological assay. Specifically, random peptide strands may be displayed on the flow cell 152, such as in step/operation 1624, and screened against antibody-containing serum samples from a patient. Following that, the imaging instrument 100 may be used to observe the flow cell 152, and based on which peptides may bind with antibodies in the serum samples and given sequencing data, an entire immune history for a patient may be identified. Therefore, various example embodiments of the present disclosure may be used for serology surveillance.

### IV. CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for determining molecular properties of at least one molecule, the method comprising:
generating a molecule library comprising the at least one molecule;
generating a focused library by filtering the molecule library, the focused library comprising the at least one molecule;
attaching the focused library to a flow cell of an imaging instrument;
controlling a fluid control module of the imaging instrument to incubate the flow cell with a fluid comprising labelled target molecules;
operating a light source module and an imaging module of the imaging instrument in a selected operational mode such that the light source module illuminates the flow cell and the imaging module captures light generated by fluorescence by the labelled target molecules within the flow cell;
generating imaging data based on the captured light; and
analyzing the imaging data to determine the molecular properties of the at least one molecule.

2. The method of claim 1, wherein the at least one molecule is configured for screening through DNA templated molecule synthesis.

3. The method of claim 2, where in the focused library comprises one of the following types: (a) cDNA, (b) mRNA, (c) peptide, or (d) DNA conjugated nanomaterials.

4. The method of claim 2, wherein the DNA templated molecule synthesis comprises one of: (a) cDNA display, (b) mRNA display, (c) ribosome display, (d) particle display, or (e) DNA conjugation on nanomaterials.

5. The method of claim 1, further comprising generating a database storing the determined molecular properties of the at least one molecule in association with genotype and phenotype information for the at least one molecule.

6. The method of claim 1, wherein the light source module and the imaging module are operated in one of (a) a Fourier ptychography (FP) operational mode, (b) a light sheet operational mode, or (c) a time delay imaging operational mode and the sampling rate of the imaging module is less than one minute.

7. The method of claim 6, wherein the determined molecular properties comprise at least one non-equilibrium property.

8. The method of claim 7, wherein the at least one non-equilibrium property comprises at least one of (a) an association rate for the at least one molecule and a target molecule or (b) a disassociation rate for the at least one molecule and the target molecule.

9. The method of claim 6, wherein operating the imaging module in one of (a) the FP operational mode or (b) the light sheet operational mode and with the sampling rate of the imaging module being less than one minute comprises capturing light emitted from the flow cell with an array of photodetectors, each photodetector of the array of photodetectors receiving a portion of the light emitted by the flow cell that was conditioned by a corresponding lens of a lens array, each lens of the lens array configured to sample a distinct spatial range of light coming from a corresponding distinct portion of the flow cell.

10. The method of claim 9, wherein each photodetector of the array of photodetectors generates a partial image such that the array of photodetectors generates a plurality of partial images that were generated simultaneously and the method further comprises analyzing the plurality of partial images via an image processing pipeline to stitch together the partial images in Fourier space and produce a complex sample image.

11. The method of claim 1, wherein the imaging data comprises first imaging data captured under first environmental conditions within the flow cell and second imaging data captured under second environmental conditions within the flow cell, wherein the first environmental conditions differs from the second environmental conditions by at least one corresponding aspect.

12. The method of claim 11, wherein the first environmental conditions are defined by a first set of aspects comprising at least one of (a) a first temperature, (b) a first pH, or (c) a first molecular composition of a fluid within the flow cell and the second environmental conditions are defined by a second set of aspects comprising at least one of (a) a second temperature, (b) a second pH, or (c) a second molecular composition of the fluid within the flow cell.

13. The method of claim 11, wherein the first environmental conditions and the second environmental conditions are controlled by at least one of the fluid control modules of the imaging instrument or a temperature control module of the imaging instrument.

14. The method of claim 1, wherein the molecule library is filtered to form the focused library based on functional selection of molecules within the molecule library.

15. The method of claim 14, wherein the functional selection of molecules comprises at least one of (a) positive selection of molecules or (b) negative selection of molecules.

16. The method of claim 1, wherein molecular properties are simultaneously determined for a plurality of molecules, the plurality of molecules comprising at least two chemically distinct molecules.

17. The method of claim 1, wherein a non-transitory computer-readable memory stores a location within the flow cell at which the at least one molecule is attached in association with a sequence corresponding to the at least one molecule.

18. An imaging instrument comprising:
a flow cell;
a fluid control module configured to control a flow of fluid through the flow cell;
a light source module configured to illuminate at least a portion of the flow cell;
a temperature control module configured to control a temperature within the flow cell;
an imaging module configured to capture light emitted from the flow cell; and
a controller configured to control operation of the fluid control module, light source module, temperature control module, and imaging module, and to receive signals corresponding to light captured by the imaging module, wherein the controller is configured to operate the imaging instrument in at least one operational mode that enables a sampling rate by the imaging module of less than one minute.

19. The imaging instrument of claim 18, wherein the controller is further configured to control operation of the flow cell, the fluid control module, the light source module, and the temperature control module, to cause the imaging instrument to perform:
control operation of the fluid control module of the imaging instrument to incubate the flow cell with a fluid comprising labelled target molecules, wherein a library of molecules is attached to the flow cell, the library of molecule comprising at least one molecule;
operate the light source module and the imaging module of the imaging instrument in a selected operational mode such that the light source module illuminates the flow cell and the imaging module captures light generated by fluorescence by the labelled target molecules within the flow cell;
generate imaging data based on the captured light; and
analyze the imaging data or provide the imaging data for analysis to determine the molecular properties of the at least one molecule.

20. The imaging instrument of claim 19, wherein generating the imaging data comprises:
controlling operation of at least one of the fluid control module or the temperature module to provide first environmental conditions within the flow cell;
generating first imaging data while the flow cell is experiencing the first environmental conditions;
controlling operation of at least one of the fluid control module or the temperature module to provide second environmental conditions within the flow cell; and
generating second imaging data while the flow cell is experiencing the second environmental conditions, wherein the first environmental conditions differs from the second environmental conditions by at least one corresponding aspect.
